(19) United States
(12) Patent Application Publication
(10) Pub. No.: US 2026/0167643 A1
(43) Pub. Date: Jun. 18, 2026

(54) COMPOUND, COMPOSITION, SURFACE TREATMENT AGENT, ARTICLE, AND METHOD FOR MANUFACTURING ARTICLE

(71) Applicant: AGC Inc., Tokyo (JP)

(72) Inventor: Koki WATANABE, Tokyo (JP)

(73) Assignee: AGC Inc., Tokyo (JP)

(21) Appl. No.: 19/530,566

(22) Filed: Feb. 5, 2026

Related U.S. Application Data
(63) Continuation of application No. PCT/JP2024/028532, filed on Aug. 8, 2024.

(30) Foreign Application Priority Data
Aug. 9, 2023 (JP) 2023-130267

(51) Int. Cl. C07D 487/04; C07D 495/04; C08G 77/388

(52) U.S. Cl. CPC C07D 487/04; C07D 495/04; C08G 77/388

(57) ABSTRACT

A new compound capable of forming a surface-treated layer having excellent light stability is provided.

A compound reprsented by below-shown Formula (1) or (2).

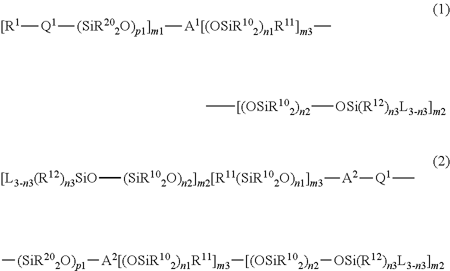

Note that each symbol in the formulas is the same as that described in the specification.

### INCORPORATION BY REFERENCE

This application is based upon and claims the benefit of priority from Japanese Patent Application 2023-130267 filed on Aug. 9, 2023, and PCT application No. PCT/JP2024/028532 filed on Aug. 8, 2024, the disclosure of which is incorporated herein in its entirety by reference.

### BACKGROUND

The present disclosure relates to a compound, a composition, a surface treatment agent, an article, and a method for manufacturing an article.

In recent years, there has been a demand for a technique for preventing the surface of an article from being stained with fingerprints, and a technique for facilitating the removal of dirt in order to improve properties of the article such as its appearance and visibility. As a specific method, a method for treating the surface of an article by using a surface treatment agent is known.

For example, Patent Literature 1 discloses a specific siloxane group-containing silane compound having a divalent linear organopolysiloxane group and a hydrolyzable silyl group.
Patent Literature 1: International Patent Publication No. WO2023/017830

### SUMMARY

It has been desired to further improve surface treatment agents in view of the tolerance to light and the like.

The present disclosure has been made in view of the above-described circumstances, and an object thereof is to provide a new compound capable of forming a surface-treated layer having excellent light stability, a composition and a surface treatment agent comprising such a compound, an article including a surface-treated layer having excellent light stability, and a method for manufacturing such an article.

The present disclosure includes the following aspects.

[1]

A compound represented by below-shown Formula (1) or Formula (2)

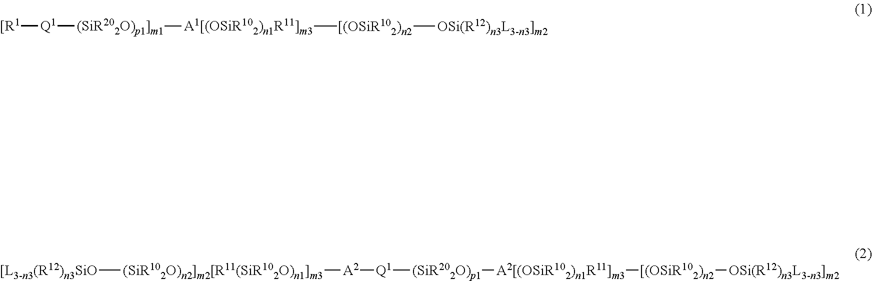

wherein
R¹ is each independently (R¹¹¹)₃Si—, a monovalent cyclic polysiloxane residue, or a monovalent cage-like polysiloxane residue,
R¹¹¹ is each independently a hydrocarbon group or a trialkylsilyloxy group, and Q¹ is each independently an oxygen atom or an alkylene group,
A¹ is a silicon atom, a cyclic organopolysiloxane residue, or a cage-like organopolysiloxane residue, and is a group having a valence of (m1+m2+m3),
A² is a silicon atom, a cyclic organopolysiloxane residue, or a cage-like organopolysiloxane residue, and is a group having a valence of (1+m2+m3),
R¹⁰ is each independently a hydrocarbon group,
R¹¹ is each independently a hydrocarbon group,
R¹² is each independently a hydrocarbon group,
R²⁰ is each independently a hydrocarbon group,
p1 is a number of 1 to 500,
L is each independently a hydrolyzable group, a group having a hydrolyzable group, or a hydroxyl group,
n1 is each independently an integer of 0 to 6,
n2 is each independently an integer of 0 to 6,
n3 is an integer of 0 to 2,
m1 is an integer of 1 or greater,
m2 is an integer of 2 or greater, and
m3 is an integer of 0 or greater.
   [2]

The compound according to Item [1], wherein Q¹ is an oxygen atom.

[3]

The compound according to Item [1] or [2], wherein m1 is an integer of 1 to 6.

[4]

The compound according to any one of Items [1] to [3], wherein m2 is an integer of 2 to 6.

[5]

The compound according to any one of Items [1] to [4], wherein m3 is 0 or 1.

[6]

The compound according to any one of Items [1] to [5], wherein A¹ is a residue obtained by excluding (m1+m2+m3) pieces of R from below-shown Formula (A1), Formula (A2), or Formula (A3)

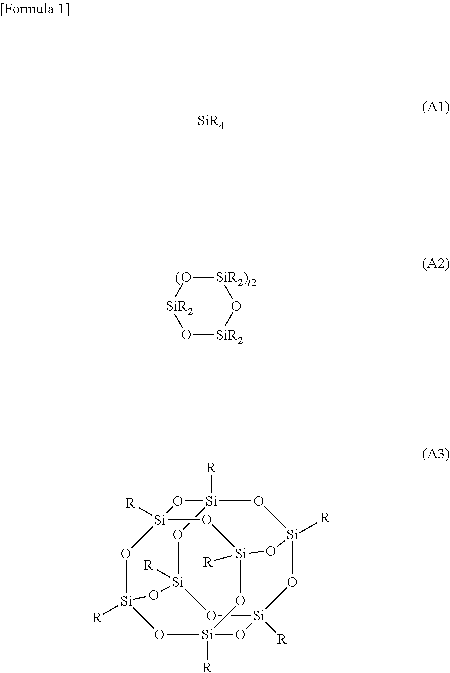

where
R is each independently [R¹-Q¹-(SiR²⁰₂O)ₚ₁], [(OSiR¹⁰₂)ₙ₁R¹¹], [(OSiR¹⁰₂)ₙ₂—OSi(R¹²)ₙ₃L₃₋ₙ₃], or a hydrocarbon group, and
t2 is an integer of 1 to 4.
   [7]

The compound according to any one of Items [1] to [5], wherein A² is each independently a residue obtained by excluding (1+m2+m3) pieces of R from above-shown Formula (A1), Formula (A2), or Formula (A³)
where
R is each independently a hydrocarbon group, or indicates a position of a bond with [(OSiR¹⁰₂)ₙ₁R¹¹] or [(OSiR¹⁰₂)ₙ₂—OSi(R¹²)ₙ₃L₃₋ₙ₃] in Formula (2), and
t2 is an integer of 1 to 4.
   [8]

A composition comprising a compound according to any one of Items [1] to [7] and a liquid medium.

[9]

A surface treatment agent comprising a compound according to any one of Items [1] to [7].

[10]

A surface treatment agent comprising a compound according to any one of Items [1] to [7] and a liquid medium.

[11]

A method for manufacturing an article including a surface-treated layer formed on a substrate by performing a surface treatment on the substrate by using a surface treatment agent comprising a compound according to any one of Items [1] to [7].

[12]

An article comprising a substrate, and a surface-treated layer disposed on the substrate, a surface of the surface-treated layer being treated with a surface treatment agent comprising a compound according to any one of Items [1] to [7].

[13]

The article according to Item [12], wherein the article is an optical member.

[14]

The article according to Item [12] or [13], wherein the article is a display or a touch panel.

[15]

A method for manufacturing an article including a surface-treated layer formed on a substrate by performing a surface treatment on the substrate by using a surface treatment agent comprising a compound according to any one of Items [1] to [7] and a liquid medium.

[16]

An article comprising a substrate, and a surface-treated layer disposed on the substrate, a surface of the surface-treated layer being treated with a surface treatment agent comprising a compound according to any one of Items [1] to [7] and a liquid medium. 15

[17]

The article according to Item [16], wherein the article is an optical member.

[18]

The article according to Item [16] or [17], wherein the article is a display or a touch panel.

According to an embodiment of the present disclosure, it is possible to provide a new compound capable of forming a surface-treated layer having excellent light stability, a composition and a surface treatment agent comprising such a compound, an article including a surface-treated layer having excellent light stability, and a method for manufacturing such an article.

The above and other objects, features and advantages of the present disclosure will become more fully understood from the detailed description given hereinbelow.

### DESCRIPTION OF EMBODIMENTS

A numerical value range specified by using “-” in the specification of the present disclosure includes numerical values before and after “-” as a lower limit value and an upper limit value, respectively, of the range.

In numerical ranges described in a stepwise manner in the present specification, the upper or lower limit value of one numerical range may be replaced with the upper or lower limit value of another numerical range described in a stepwise manner. Further, in numerical ranges described in a stepwise manner in the present specification, the upper or lower limit value of a numerical range may be replaced with values shown in Examples.

In the specification of the present disclosure, the “surface-treated layer” refers to a layer that is formed on the surface of a substrate by a surface treatment.

In this specification, “Me” may represent a methyl group, and “Et” may represent an ethyl group.

In this specification, when a compound or a group is represented by a specific formula (X), the compound or the group represented by this formula (X) may be expressed as a compound (X) or a compound X, and a group (X) or a group X, respectively.

Note that when the same symbols are present in one chemical formula, these same symbols may represent structures same as each other, or may represent structures different from each other within a specified range.

The bonding direction of a divalent group shown in the specification of the present disclosure is not limited to any particular directions unless otherwise specified. For example, when Y is —COO— in a compound represented by a formula “X—Y—Z”, Y may be —CO—O— or —O—CO—. Further, the aforementioned compound may be “X—CO—O—Z” or “X—O—CO—Z”.

### [Compound]

A compound according to the present disclosure is a compound represented by the below-shown Formula (1) or (2).

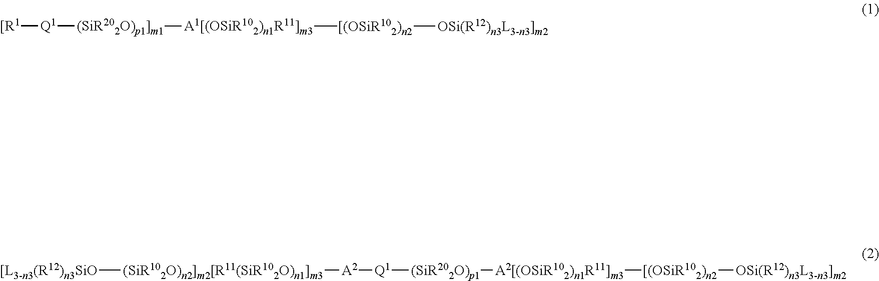

Where
R¹ is each independently (R¹¹¹)₃Si—, a monovalent cyclic polysiloxane residue, or a monovalent cage-like polysiloxane residue,
R¹¹¹ is each independently a hydrocarbon group or a trialkylsilyloxy group, and Q¹ is each independently an oxygen atom or an alkylene group,
A¹ is a silicon atom, a cyclic organopolysiloxane residue, or a cage-like organopolysiloxane residue, and is a group having a valence of (m1+m2+m3),
A² is a silicon atom, a cyclic organopolysiloxane residue, or a cage-like organopolysiloxane residue, and is a group having a valence of (1+m2+m3),
R¹⁰ is each independently a hydrocarbon group,
R¹¹ is each independently a hydrocarbon group,
R¹² is each independently a hydrocarbon group,
R²⁰ is each independently a hydrocarbon group,
p1 is a number of 1 to 500,
L is each independently a hydrolyzable group, a group having a hydrolyzable group, or a hydroxyl group,
n1 is each independently an integer of 0 to 6,
n2 is each independently an integer of 0 to 6,
n3 is an integer of 0 to 2,
m1 is an integer of 1 or greater,
m2 is an integer of 2 or greater, and
m3 is an integer of 0 or greater.

When a compound according to the present disclosure is used as a surface treatment agent, a surface-treated layer having excellent light stability can be formed. Although the reason for this feature is not clear, it is presumed as follows.

Since the compound according to the present disclosure has a reactive silyl group having a high adhesive property for a substrate, a surface-treated layer which closely adheres to the surface of the substrate can be formed. Further, it is considered that since the linking group A which bonds the reactive silyl group to the organopolysiloxane residue is a silicon atom, a cyclic organopolysiloxane residue, or a cage-like organopolysiloxane residue, a surface-treated layer of which the light stability is superior to those of compounds having other types of linking groups can be formed.

A compound according to the present disclosure will be described hereinafter in detail.

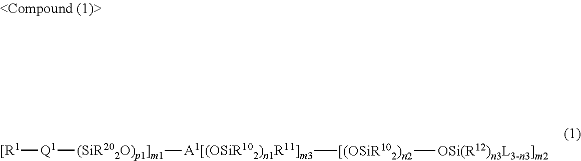

In Formula (1),
R¹ is each independently (R¹¹¹)₃Si—, a monovalent cyclic polysiloxane residue, or a monovalent cage-like polysiloxane residue,
R¹¹¹ is each independently a hydrocarbon group or a trialkylsilyloxy group, and Q¹ is each independently an oxygen atom or an alkylene group,
A¹ is a silicon atom, a cyclic organopolysiloxane residue, or a cage-like organopolysiloxane residue, and is a group having a valence of (m1+m2+m3),
R¹⁰ is each independently a hydrocarbon group,
R¹¹ is each independently a hydrocarbon group,
R¹² is each independently a hydrocarbon group,
R²⁰ is each independently a hydrocarbon group,
p1 is a number of 1 to 500,
L is each independently a hydrolyzable group, a group having a hydrolyzable group, or a hydroxyl group,
n1 is each independently an integer of 0 to 6,
n2 is each independently an integer of 0 to 6,
n3 is an integer of 0 to 2,
m1 is an integer of 1 or greater,
m2 is an integer of 2 or greater, and
m3 is an integer of 0 or greater.
R¹ in Formula (1) is (R¹¹¹)₃Si—, a monovalent cyclic polysiloxane residue, or a monovalent cage-like polysiloxane residue.

In (R¹¹¹)₃Si— of R¹, R¹¹¹ is a hydrocarbon group or a trialkylsilyloxy group.

The hydrocarbon group in R¹¹¹ is preferably an alkyl group or an aryl group, and more preferably an alkyl group.

The alkyl group may be any of a linear alkyl group, a branched alkyl group, and a cyclic alkyl group, and is preferably a linear alkyl group. The number of carbon atoms of the alkyl group is preferably 1 to 10, more preferably 1 to 8, and still more preferably 1 to 4. The same applies to the alkyl group in the trialkylsilyloxy group.

Examples of the trialkylsilyloxy group of R¹¹¹ include a group represented by (R¹¹²)₃SiO—, and R¹¹² is each independently a hydrocarbon group. Examples of the hydrocarbon group of R¹¹² include groups similar to those of R¹¹¹.

Specific examples of R¹¹¹ include a methyl group, an ethyl group, and a tert-butyl group. Further, R¹¹¹ is preferably a methyl group or an ethyl group, and more preferably a methyl group.

The cyclic organopolysiloxane group in R¹ is preferably a group represented by the below-shown Formula (B1).

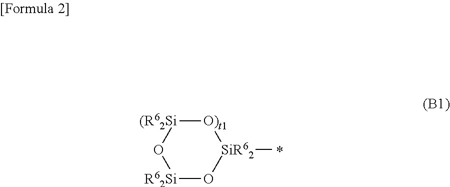

Note that R⁶ is each independently a hydrocarbon group, a hydrocarbon group having a substituent, or a trialkylsilyloxy group, and t1 is a number of 1 to 4.

The hydrocarbon group in R⁶ is preferably an alkyl group or an aryl group, and more preferably an alkyl group.

The alkyl group may be any of a linear alkyl group, a branched alkyl group, and a cyclic alkyl group, and is preferably a linear alkyl group. The number of carbon atoms of the alkyl group is preferably 1 to 10, more preferably 1 to 8, and still more preferably 1 to 4. The same applies to the alkyl group in the trialkylsilyloxy group.

Regarding the hydrocarbon group having a substituent, examples of the substituent include a halogen atom, a hydroxyl group, an alkoxy group, a trialkylsilyloxy group, a trialkylsilyl group, an amino group, a nitro group, a cyano group, a sulfonyl group, and a trifluoromethyl group.

Examples of the trialkylsilyloxy group of R⁶ include groups similar to those of R¹¹¹.

Specific examples of R⁶ include a methyl group, an ethyl group, and a tert-butyl group. Further, R⁶ is preferably a methyl group or an ethyl group, and more preferably a methyl group.

Specific examples of the cyclic organopolysiloxane group include groups shown below.

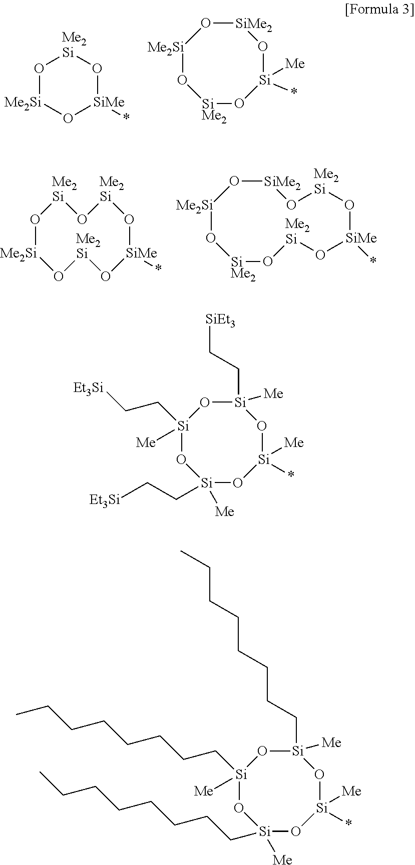

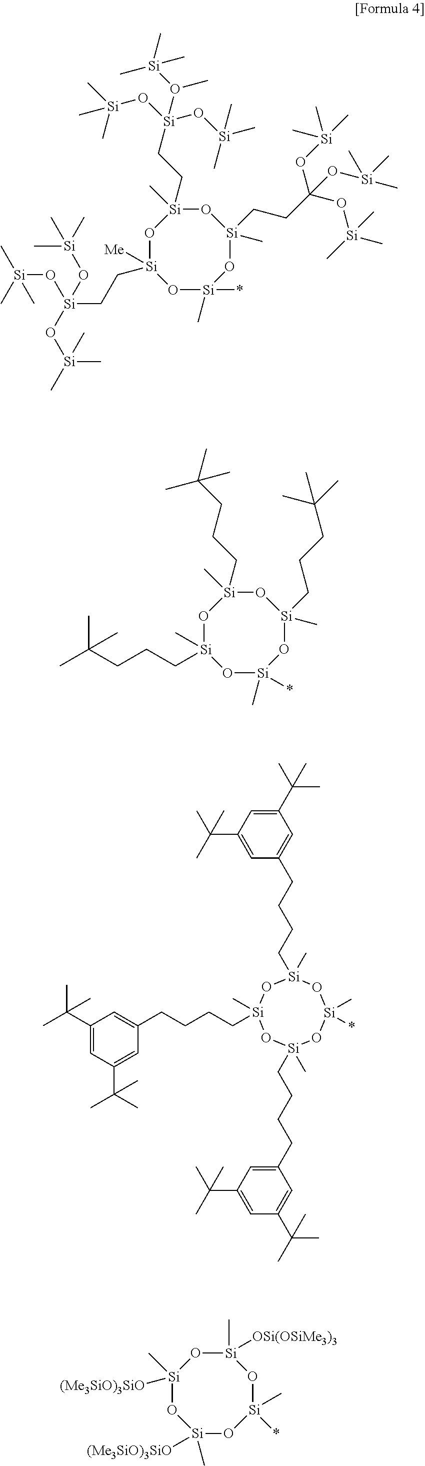

The cage-like organopolysiloxane group is preferably a group represented by the below-shown Formula (B2).

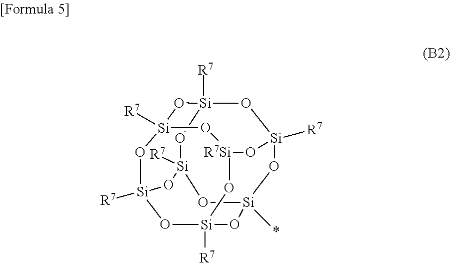

Note that R⁷ is each independently a hydrocarbon group, a hydrocarbon group having a substituent, or a trialkylsilyloxy group.

The hydrocarbon group in R⁷ is preferably an alkyl group or an aryl group, and more preferably an alkyl group.

The alkyl group may be any of a linear alkyl group, a branched alkyl group, and a cyclic alkyl group, and is preferably a linear alkyl group. The number of carbon atoms of the alkyl group is preferably 1 to 10, more preferably 1 to 8, and still more preferably 1 to 4. The same applies to the alkyl group in the trialkylsilyloxy group.

Regarding the hydrocarbon group having a substituent, examples of the substituent include a halogen atom, a hydroxyl group, an alkoxy group, a trialkylsilyloxy group, a trialkylsilyl group, an amino group, a nitro group, a cyano group, a sulfonyl group, and a trifluoromethyl group.

Examples of the trialkylsilyloxy group of R⁷ include groups similar to those of R¹¹¹.

Specific examples of R⁷ include a methyl group, an ethyl group, and a tert-butyl group. Further, R⁷ is preferably a methyl group or an ethyl group, and more preferably a methyl group.

Specific examples of the cage-like organopolysiloxane group include groups shown below.

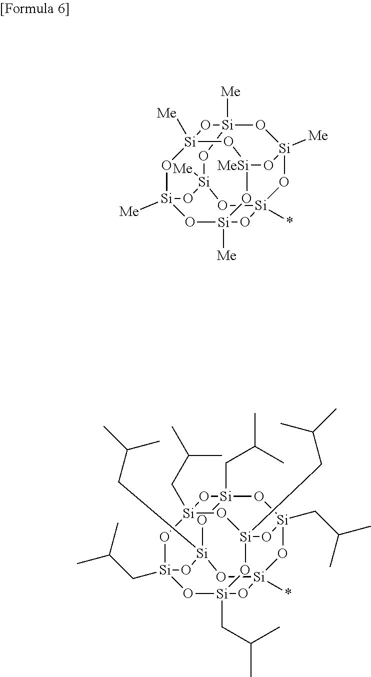

R¹ is preferably (R¹¹¹)₃Si— in view of the light stability, the liquid repellency, and the ease of the synthesis, and in particular, is preferably (CH₃)₃Si— or (CH₃SiO)₃Si—.

Q¹ in Formula (1) is an oxygen atom or an alkylene group.

Examples of the alkylene group of Q¹ include a linear alkylene group, a branched alkylene group, and a cyclic alkylene group. Further, the alkylene group is preferably a linear alkylene group in view of the light stability. Further, the number of carbon atoms of the alkylene group is preferably 1 to 30, more preferably 1 to 12, and still more preferably 1 to 6. Specific examples of alkylene groups include a methylene group, an ethylene group, a propylene group, a butylene group, and a hexylene group.

In view of the light stability and the liquid repellency, Q¹ is preferably an oxygen atom or a linear alkylene group having a number of carbon atoms of 1 to 6, more preferably an oxygen atom, a methylene group, or an ethylene group, and still more preferably an oxygen atom.

(SiR²⁰₂O)ₚ₁ in Formula (1) represents a linear organopolysiloxane; R²⁰ is a hydrocarbon group; and p1 is a number of 1 to 500.

The hydrocarbon group in R²⁰ is preferably an alkyl group or an aryl group. The alkyl group may be any of a linear alkyl group, a branched alkyl group, and a cyclic alkyl group, and is preferably a linear alkyl group or a branched alkyl group. The number of carbon atoms of the alkyl group is preferably 1 to 10, more preferably 1 to 8, and still more preferably 1 to 4. Specific examples of the alkyl group include a methyl group, an ethyl group, and a tert-butyl group. Further, specific examples of aryl groups include a phenyl group and a naphthyl group.

In particular, in view of the light stability and the liquid repellency, R²⁰ is preferably a methyl group, an ethyl group, a tert-butyl group, or a phenyl group, more preferably a methyl group or an ethyl group, and still more preferably a methyl group.

Further, p1 is a number of 1 to 500, and is preferably a number of 1 to 300 and more preferably a number of 1 to 200 because the liquid repellency and the durability of the compound become excellent.

(OSiR¹⁰₂) in Formula (1) represents a linear organopolysiloxane branched from the linking group A¹, and R¹⁰ is a hydrocarbon group. The hydrocarbon group in R¹⁰ is similar to that in the above-described R²⁰, and its preferred forms are also similar to those described above.

R¹¹ in Formula (1) is a hydrocarbon group and constitutes one end.

Examples of the hydrocarbon group in R¹¹ include an alkyl group and an aryl group. The alkyl group may be any of a linear alkyl group, a branched alkyl group, and a cyclic alkyl group, and is preferably a linear alkyl group or a branched alkyl group, and more preferably a linear alkyl group. Further, in view of the light stability and the liquid repellency, the hydrocarbon group in R¹¹ is more preferably an alkyl group having a number of carbon atoms of 6 or greater, and more preferably an alkyl group having a number of carbon atoms of 10 to 30.

Each of n1 and n2 in Formula (1) represents the number of repetitions of (SiR²⁰₂O) branched from A¹, and is each independently an integer of 0 to 6, preferably 0 to 3, and more preferably 1 or 2.

Si(R¹²)ₙ₃L₃₋ₙ₃ in Formula (1) represents a reactive silyl group; R¹² is a hydrocarbon group; L is a hydrolyzable group, a group having a hydrolyzable group, or a hydroxyl group; and n3 is an integer of 0 to 2.

Examples of the hydrocarbon group of R¹² include an alkyl group, a cycloalkyl group, an alkenyl group, and an allyl group. Further, in view of the ease of the synthesis and the like, the hydrocarbon group is preferably a saturated hydrocarbon group and more preferably an alkyl group. The number of carbon atoms of R¹² is preferably 1 to 6, more preferably 1 to 3, and still more preferably 1 or 2.

L is each independently a hydrolyzable group, a group having a hydrolyzable group, or a hydroxyl group. The hydrolyzable group is a group which becomes a hydroxyl group through a hydrolysis reaction.

That is, the hydrolyzable silyl group represented by Si-L becomes a silanol group represented by Si—OH through a hydrolysis reaction. Such silanol groups further react with each other and thereby form a Si—O—Si bond. Further, such a silanol group has a dehydration condensation reaction with a silanol group derived from an oxide present on the surface of the substrate, and thereby can form a Si—O—Si bond.

Examples of hydrolyzable groups include an alkoxy group, an aryloxy group, a halogen atom, an acyl group, an acyloxy group, and an isocyanato group (—NCO).

The number of carbon atoms of the alkoxy group is preferably 1 to 6 and more preferably 1 to 4. The aryloxy group is preferably an aryloxy group having a number of carbon atoms of 3 to 10. However, the aryl group of the aryloxy group may be a heteroaryl group. The halogen atom is preferably a chlorine atom. The acyl group is preferably an acyl group having a number of carbon atoms of 1 to 6. The acyloxy group is preferably an acyloxy group having a number of carbon atoms of 1 to 6. Further, the alkylene oxide-modified alkoxy group is preferably a group represented by —(O—R⁶¹)ₙ₁₁—L¹ (where R⁶¹ is an alkylene group having a number of carbon atoms of 1 to 10; L¹ is an alkoxy group having a number of carbon atoms of 1 to 6; and n11 is an integer of 1 to 6). In particular, R⁶¹ is preferably an alkylene group having a number of carbon atoms of 1 to 6, and n11 is preferably 1.

The group having a hydrolyzable group may be, for example, the group having a hydrolyzable group shown above as an example. The group having a hydrolyzable group is preferably —O-L^{A}-L^{B}. L^{A} is an alkylene group, and L^{B} is a hydrolyzable group. The number of carbon atoms of the alkylene group is preferably 1 to 10. The hydrolyzable group represented by L^{B} is synonymous with the hydrolyzable group represented by the above-described L, and its preferred forms are also the same as those described above. Specific examples of the group having a hydrolyzable group include —O—CH₂CH₂—OCH₃.

In particular, L is preferably an alkoxy group having a number of carbon atoms of 1 to 4, an alkylene oxide-modified alkoxy group, or a halogen atom in view of the ease of the manufacturing of the compound. L is preferably an alkoxy group having a number of carbon atoms of 1 to 4, and more preferably an ethoxy group or a methoxy group in view of the fact that outgassing during the application process is small and because the storage stability of the compound becomes more excellent.

n3 is an integer of 0 to 2, and is preferably 0 or 1, and more preferably 0. The presence of a plurality of L makes the adhesive property of the surface-treated layer for the substrate stronger.

When n3 is 1 or smaller, the plurality of L present in one molecule may be the same as each other or different from each other. The plurality of L are preferably the same as each other in view of the availability of raw materials and the ease of the manufacturing of the compound. When n3 is 2, the plurality of R¹² present in one molecule may be the same as each other or different from each other. The plurality of R¹² are preferably the same as each other in view of the availability of raw materials and the ease of the manufacturing of the compound.

A¹ in Formula (1) is a silicon atom, a cyclic organopolysiloxane residue, or a cage-like organopolysiloxane residue, and is a group having a valence of (m1+m2+m3). Further, m1 is an integer of 1 or greater; m2 is an integer of 2 or greater; and m3 is an integer of 0 or greater.

When A¹ is a silicon atom, A¹ is, from Formula (A1), R, and is preferably a residue obtained by excluding a group in which R is [R¹. Q¹-(SiR²⁰₂O)ₚ₁], [(OSiR¹⁰₂)ₙ₁R¹¹], or [(OSiR¹⁰₂)ₙ₂—OSi(R¹²)ₙ₃L₃₋ₙ₃].

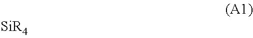

Note that R is [R¹-Q¹-(SiR²⁰₂O)ₚ₁], [(OSiR¹⁰₂)ₙ₁R¹¹], or [(OSiR¹⁰₂)ₙ₂—OSi(R¹²)ₙ₃L₃₋ₙ₃] of Formula 1, or a hydrocarbon group.

Note that “being R, and being a residue obtained by excluding a group in which R is [R¹-Q¹-(SiR²⁰₂O)ₚ₁], [(OSiR¹⁰₂)ₙ₁R¹¹], or [(OSiR¹⁰₂)ₙ₂—OSi(R¹²)ₙ₃L₃₋ₙ₃]” means that these groups explicitly shown in Formula (1) are not contained in the linking group A¹. That is, A¹ is a residue obtained by excluding (m1+m2+m3) pieces of R from Formula (A1), and the excluded R represent positions of bonds with the respective substituents in Formula (1). Note that the same applies to the below-shown Formulas (A²) and (A³).

The hydrocarbon group in R is preferably an alkyl group or an aryl group. The alkyl group may be any of a linear alkyl group, a branched alkyl group, and a cyclic alkyl group, and is preferably a linear alkyl group or a branched alkyl group. The number of carbon atoms of the alkyl group is preferably 1 to 10, more preferably 1 to 8, and still more preferably 1 to 4. Specific examples of the alkyl group include a methyl group, an ethyl group, and a tert-butyl group. Further, specific examples of aryl groups include a phenyl group and a naphthyl group.

In particular, in view of the light stability and the liquid repellency, R is preferably a methyl group, an ethyl group, a tert-butyl group, or a phenyl group, more preferably a methyl group or an ethyl group, and still more preferably a methyl group.

Note that when A¹ is a silicon atom, m1 is 1 or 2; m2 is 2 or 3; m3 is 0 or 1; and m1+m2+m3 is 4.

Specific examples of the compound according to the present disclosure in which A¹ is a silicon atom include, but are not limited to, compounds represented by the below-shown formulas.

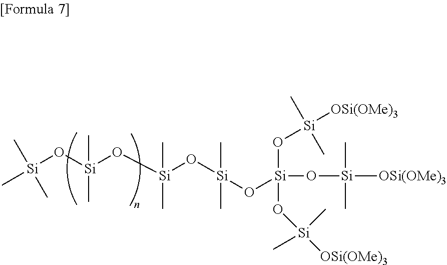

where n is a number of 1 to 500.

When A¹ is a cyclic organopolysiloxane residue, A¹ is, from the below-shown Formula (A2), R, and is preferably a residue obtained by excluding a group in which R is [R¹-Q¹-(SiR²⁰₂O)ₚ₁], [(OSiR¹⁰₂)ₙ₁R¹¹], or [(OSiR¹⁰₂)ₙ₂—OSi(R¹²)ₙ₃L₃₋ₙ₃]. That is, A¹, which is a cyclic organopolysiloxane residue, is a residue obtained by excluding (m1+m2+m3) pieces of R from Formula (A2), and the excluded R represent positions of bonds with the respective substituents in Formula (1).

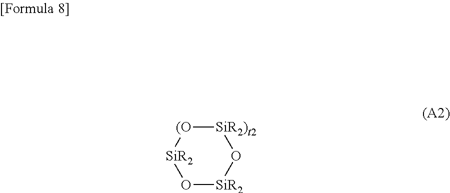

where R is similar to that in Formula (A1), and t2 is an integer of 1 to 4.

Specific examples of Formula (A2) include those shown below.

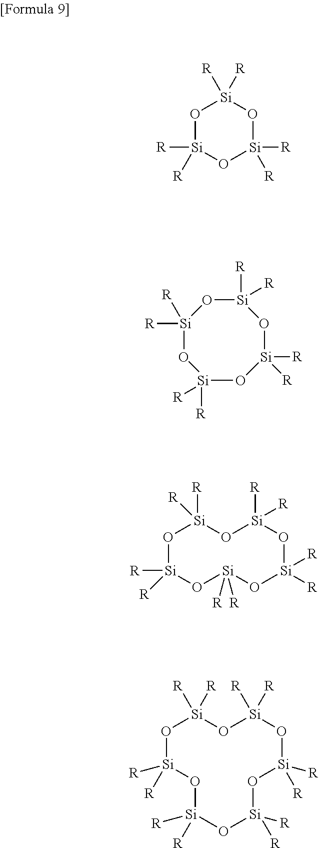

In view of the light stability, the liquid repellency, and the adhesive property for the substrate, at least one of two R bonded to one Si atom is preferably a hydrocarbon group.

Further, when A¹ is a cyclic organopolysiloxane residue, m1 is preferably 1 to 4 and more preferably 1 to 3. m2 is preferably 2 to 4 and more preferably 2 or 3. Alternatively, m3 is preferably 0 to 2 and more preferably 0 or 1.

Specific examples of the compound according to the present disclosure in which A¹ is a cyclic organopolysiloxane residue include, but are not limited to, compounds represented by the below-shown formulas.

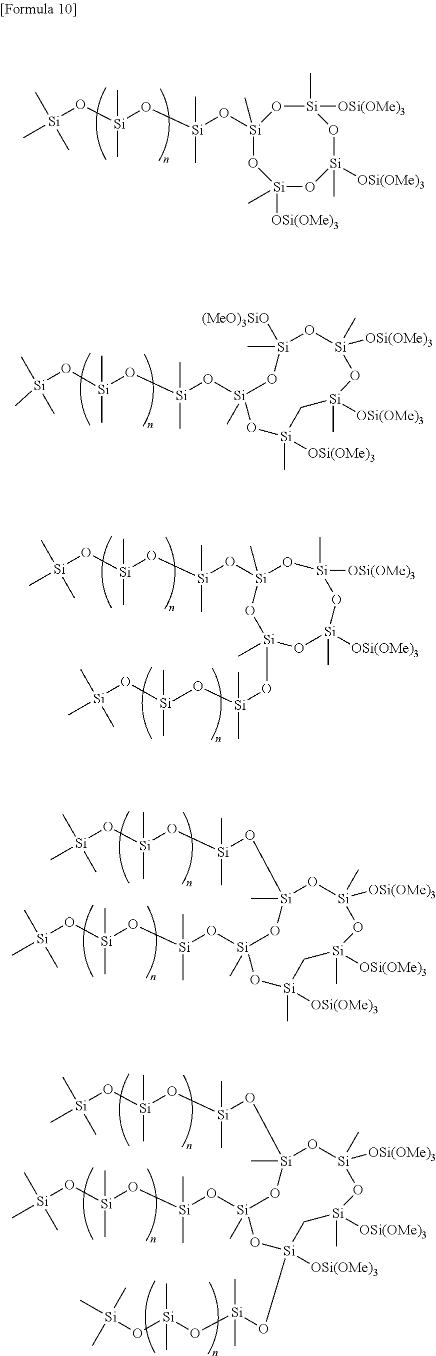

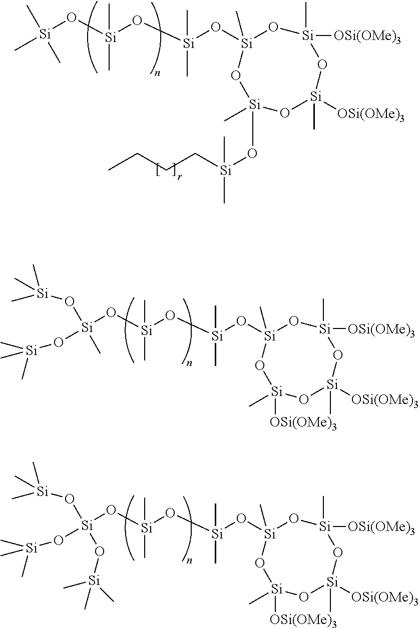

where n is a number of 1 to 500, and r is an integer of 1 to 30.

When A¹ is a cage-like organopolysiloxane residue, A¹ is, from the below-shown Formula (A3), R, and is preferably a residue obtained by excluding a group in which R is [R¹-Q¹-(SiR²⁰₂O)ₚ₁], [(OSiR¹⁰₂)ₙ₁R¹¹], or [(OSiR¹⁰₂)ₙ₂—OSi(R¹²)ₙ₃L₃₋ₙ₃]. That is, A¹, which is a cage-like organopolysiloxane residue, is a residue obtained by excluding (m1+m2+m3) pieces of R from Formula (A3), and the excluded R represent positions of bonds with the respective substituents in Formula (1).

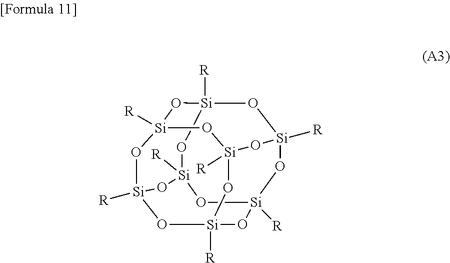

where R is similar to that in Formula (A1).

When A¹ is a cage-like organopolysiloxane residue, m1 is preferably 1 to 4 and more preferably 1 to 3. m2 is preferably 2 to 7 and more preferably 2 to 5.

Alternative, m3 is preferably 0 to 2 and more preferably 0 or 1.

Specific examples of the compound according to the present disclosure in which A¹ is a cage-like organopolysiloxane residue include, but are not limited to, compounds represented by the below-shown formulas.

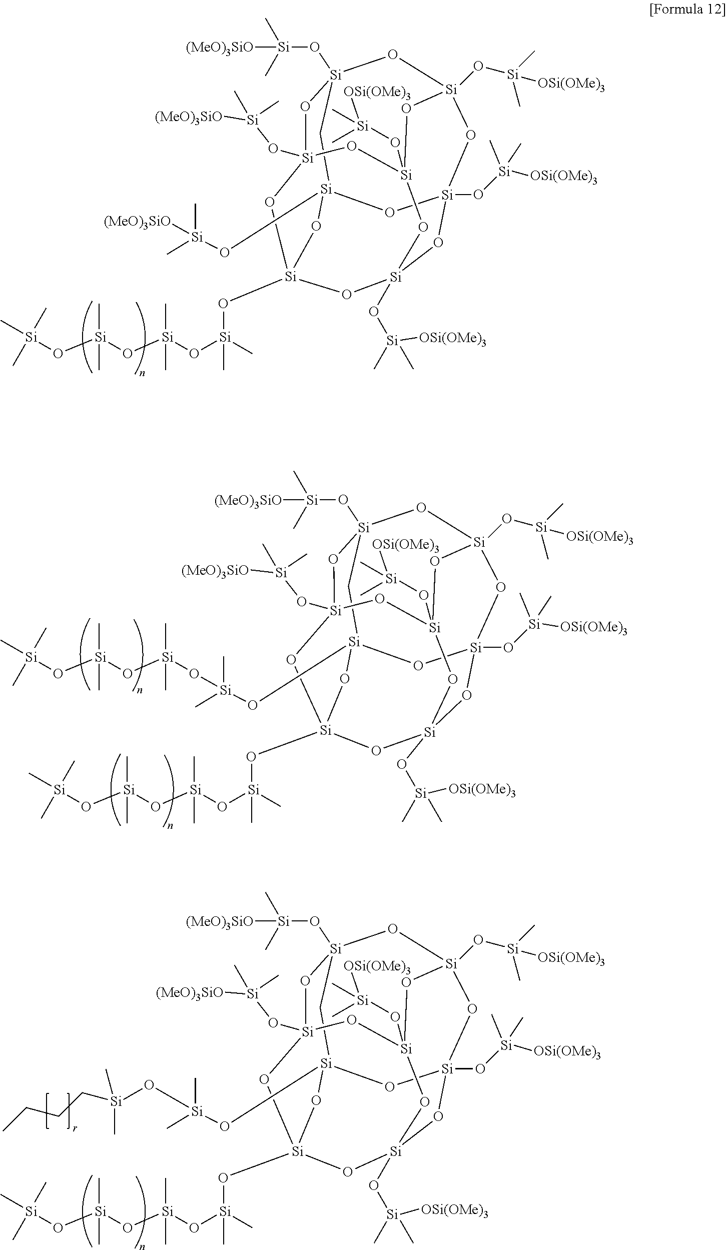

where n is a number of 1 to 500, and r is an integer of 1 to 30.

### <Compound (2)>

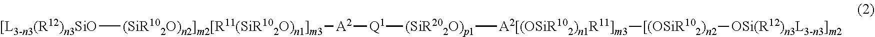

In Formula (2),
Q¹ is each independently an oxygen atom or an alkylene group,
A² is a silicon atom, a cyclic organopolysiloxane residue, or a cage-like organopolysiloxane residue, and is a group having a valence of (1+m2+m3),
R¹⁰ is each independently a hydrocarbon group,
R¹¹ is each independently a hydrocarbon group,
R¹² is each independently a hydrocarbon group,
R²⁰ is each independently a hydrocarbon group,
p1 is a number of 1 to 500,
L is each independently a hydrolyzable group, a group having a hydrolyzable group, or a hydroxyl group,
n1 is each independently an integer of 0 to 6,
n2 is each independently an integer of 0 to 6,
and n3 is an integer of 0 to 2,
m2 is an integer of 2 or greater, and
m3 is an integer of 0 or greater.

In Formula (2), Q¹, R¹⁰, R¹¹, R¹², R²⁰, p1, L, n1, n2, n3, m2, and m3 are synonymous with the respective groups in Formula (1), and their preferred forms are also the same as those described above.

A² in Formula (2) is a silicon atom, a cyclic organopolysiloxane residue, or a cage-like organopolysiloxane residue, and is a group having a valence of (1+m2+m3). Further, m2 is an integer of 2 or greater, and m3 is an integer of 0 or greater.

Examples of specific structures of A² include structures similar to those of Formulas (A¹), (A²) and (A³) in the above-described A¹. However, m1 in A¹ is replaced with 1. That is, A² is preferably each independently a residue obtained by excluding (1+m2+m3) pieces of R from Formula (A1), Formula (A2), or Formula (A3). Note that the excluded R represent positions of bonds with the respective substituents in Formula (2).

(OSiR¹⁰₂) in Formula (2) represents a linear organopolysiloxane branched from the linking group A², and R¹⁰ is a hydrocarbon group. The hydrocarbon group in R¹⁰ is similar to that in the above-described R²⁰, and its preferred forms are also similar to those described.

Each of n1 and n2 in Formula (2) represents the number of repetitions of (SiR²⁰₂O) branched from A², and is each independently an integer of 0 to 6, preferably 0 to 3, and more preferably 1 or 2.

Specific examples of the compound (2) include, but are not limited to, compounds represented by the below-shown formulas.

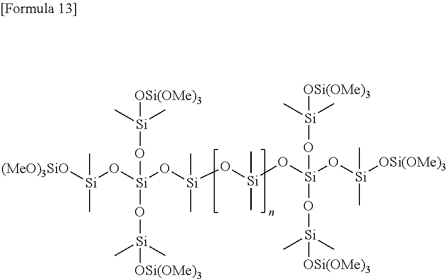

where n is a number of 1 to 500.

The number-average molecular weight (Mn) of a compound according to the present disclosure is preferably 500 to 20,000, more preferably 600 or 18,000, and still more preferably 700 or 15,000.

When Mn is 500 or larger, the abrasion resistance of the surface-treated layer becomes more excellent. When Mn is 20,000 or smaller, the viscosity can be easily adjusted within an appropriate range and the solubility is improved, so that the handling property during the film formation (i.e., during the deposition of the compound) becomes excellent.

### [Method for Manufacturing Compound]

The method for manufacturing the above-described compound disclosed herein is not limited to any particular methods. For example, the compound disclosed herein can be synthesized by adding a cyclic or cage-like organopolysiloxane corresponding to A¹ or A² to the linear organopolysiloxane comprising (SiR²⁰₂O)ₚ₁, and then introducing a reactive silyl group therein. The conditions for the reaction may be adjusted as appropriate with reference to Examples (which will be described later).

### [Composition]

It is sufficient if a composition according to the present disclosure contains a compound according to the present disclosure, and its components other than the compound according to the present disclosure are not limited to any particular components. The composition according to the present disclosure preferably contains the compound according to the present disclosure and a liquid medium. In the case where the composition according to the present disclosure contains a liquid medium, it is sufficient if the liquid medium is in a liquid state. That is, the liquid medium may be a solution or a dispersion liquid.

It is sufficient if the composition according to the present disclosure contains a compound according to the present disclosure, and the composition may also contain impurities such as by-products generated in the process for manufacturing the compound according to the present disclosure.

The content of the compound according to the present disclosure is preferably 0.001 to 40 mass %, more preferably 0.01 to 20 mass %, and still more preferably 0.1 to 10 mass % based on the total amount of the composition according to the present disclosure. In the case where the composition according to the present disclosure is used in a wet coating method, the content of the compound according to the present disclosure may be 0.01 to 10 mass %, 0.02 to 5 mass %, 0.03 to 3 mass %, or 0.05 to 2 mass % based on the total amount of the composition according to the present disclosure.

The composition according to the present disclosure may contain only one type of liquid medium, or may contain two or more types of liquid mediums.

The liquid medium is preferably an organic solvent.

Examples of organic solvents include compounds consisting solely of hydrogen atoms and carbon atoms, and compounds consisting solely of hydrogen atoms, carbon atoms, and oxygen atoms.

Specifically, examples include hydrocarbon-based organic solvents, ketone-based organic solvents, ether-based organic solvents, ester-based organic solvents, glycol-based organic solvents, and alcohol-based organic solvents.

Specific examples of hydrocarbon-based organic solvents include pentane, hexane, heptane, octane, hexadecane, isohexane, isooctane, isononane, cycloheptane, cyclohexane, bicyclohexyl, benzene, toluene, ethylbenzene, o-xylene, m-xylene, p-xylene, o-diethylbenzene, m-diethylbenzene, p-diethylbenzene, n-butylbenzene, sec-butylbenzene, and tert-butylbenzene.

Specific examples of ketone-based organic solvents include acetone, methyl ethyl ketone, methyl isobutyl ketone, diisobutyl ketone, cyclohexanone, 2-heptanone, 4-heptanone, 3,5,5-trimethyl-2-cyclohexene-1-one, 3,3,5-trimethylcyclohexanone, and isophorone.

Specific examples of ether-based organic solvents include diethyl ether, cyclopentyl methyl ether, tetrahydrofuran, and 1,4-dioxane. Specific examples of ester-based organic solvents include methyl acetate, ethyl acetate, propyl acetate, isopropyl acetate, butyl acetate, isobutyl acetate, tert-butyl acetate, amyl acetate, isoamyl acetate, ethyl 3-ethoxypropionate, ethyl lactate, ethylene glycol monobutyl ether acetate, diethylene glycol monobutyl ether acetate, propylene glycol monomethyl ether acetate, dipropylene glycol methyl ether acetate, 3-methoxy-3-methylbutyl acetate, 3-methoxybutyl acetate, propylene glycol monomethyl acetate, propylene glycol dimethyl acetate, ethylene glycol monoethyl ether acetate, ethylene glycol monomethyl ether acetate, diethylene glycol monoethyl ether acetate, cyclohexanol acetate, propylene glycol diacetate, propylene glycol monomethyl ether acetate, propylene glycol monomethyl ether propionate, propylene glycol monoethyl ether acetate, propylene glycol monobutyl ether acetate, propylene glycol monopropyl ether acetate, propylene glycol diacetate, dipropylene glycol-methyl ether acetate, 1,3-butylene glycol diacetate, 1,4-butanediol diacetate, 1,3-butylene glycol diacetate, 1,6-hexanediol diacetate, γ-butyrolactone, triacetin, and 2,2,4-trimethyl-1,3-pentanediol monoisobutyrate.

Specific examples of glycol-based organic solvents include ethylene glycol, ethylene glycol monobutyl ether, diethylene glycol monobutyl ether, triethylene glycol monobutyl ether, tetraethylene glycol monobutyl ether, ethylene glycol monohexyl ether, diethylene glycol monohexyl ether, ethylene glycol mono-2-ethylhexyl ether, diethylene glycol mono-2-ethylhexyl ether, propylene glycol monomethyl ether, propylene glycol monoethyl ether, propylene glycol monobutyl ether, propylene glycol monopropyl ether, ethylene glycol monoisopropyl ether, ethylene glycol monoethyl ether, ethylene glycol mono tert-butyl ether, ethylene glycol monopropyl ether, ethylene glycol monomethyl ether, diethylene glycol monoisopropyl ether, diethylene glycol monomethyl ether, dipropylene glycol monoethyl ether, dipropylene glycol monobutyl ether, dipropylene glycol monopropyl ether, dipropylene glycol monomethyl ether, dipropylene glycol monopropyl ether, dipropylene glycol monomethyl ether, tripropylene glycol monobutyl ether tripropylene glycol monomethyl ether, propylene glycol monophenyl ether, 1,3-butylene glycol, propylene glycol n-propyl ether, propylene glycol n-butyl ether, diethylene glycol monoethyl ether, dipropylene glycol n-propyl ether, dipropylene glycol n-butyl ether, tripropylene glycol methyl ether, tripropylene glycol n-butyl ether, ethylene glycol dimethyl ether, ethylene glycol diethyl ether, diethylene glycol diethyl ether, diethylene glycol dimethyl ether, diethylene glycol dimethyl ether, dipropylene glycol dimethyl ether, diethylene glycol dibutyl ether, tetraethylene glycol dimethyl ether, triethylene glycol dimethyl ether, and polyethylene glycol dimethyl ether.

Specific examples of alcohol-based organic solvents include methanol, ethanol, 1-propanol, isopropyl alcohol, n-butanol, diacetone alcohol, isobutanol, sec-butanol, tert-butanol, pentanol, 3-methyl-1,3-butanediol, 1,3-butanediol, 1,3-butylene glycol, octanediol, 2,4-diethylpentanediol, butylethylpropanediol, 2-methyl-1,3-propanediol, 4-hydroxy-4-methyl-2-pentanone, 2-ethyl-1-hexanol, 3,5,5-trimethyl-1-hexanol, isodecanol, isotridecanol, 3-methoxy-3-methyl-1-butanol, 2-methoxybutanol, 3-methoxybutanol, cyclohexanol, furfuryl alcohol, tetrahydrofurfuryl alcohol, benzyl alcohol, and methylcyclohexanol.

Further, examples of organic solvents include halogen-based organic solvents, nitrogen-containing compounds, sulfur-containing compounds, siloxane compounds, and fluorine-containing organic solvents.

Specific examples of halogen-based organic solvents include dichloromethane, chloroform, carbon tetrachloride, dichloroethane, chlorobenzene, o-chlorotoluene, m-chlorotoluene, p-chlorotoluene, m-dichlorobenzene, and 1,2,3-trichloropropane.

Examples of nitrogen-containing compounds include nitrobenzene, acetonitrile, benzonitrile, N, N-dimethylformamide, N,N-dimethylacetamide, N-methylpyrrolidone, and 1,3-dimethyl-2-imidazolidinone.

Examples of sulfur-containing compounds include carbon disulfide and dimethyl sulfoxide.

Examples of siloxane compounds include hexamethyldisiloxane, hexaethyldisiloxane, octamethyltrisiloxane, octaethyltrisiloxane, hexamethylcyclotrisiloxane, hexaethylcyclotrisiloxane, octamethylcyclotetrasiloxane, octaethylcyclotetrasiloxane, and decamethyltetrasiloxane.

Examples of fluorine-containing organic solvents include polyfluoro aromatic hydrocarbons (e.g., 1,3-bis(trifluoromethyl)benzene); polyfluoro aliphatic hydrocarbons (e.g., C₆F₁₃CH₂CH₃ (e.g., Asahikrin (Registered Trademark) AC-6000 manufactured by AGC Inc.)), 1,1,2,2,3,3,4-heptafluorocyclopentane (e.g., Zeorora (Registered Trademark) H manufactured by Zeon Corporation); hydrofluoroethers (HFE) (e.g., alkyl perfluoroalkyl ethers (perfluoroalkyl group and alkyl group may be linear chain or branched) such as perfluoropropyl methyl ether (C₃F₇OCH₃) (e.g., Novec (Registered Trademark) 7000 manufactured by 3M Japan Limited), perfluorobutyl methyl ether (C₄F₉OCH₃) (e.g., Novec (Registered Trademark) 7100 manufactured by 3M Japan Limited), perfluorobutyl ethyl ether (C₄F₉OC₂H₅) (e.g., Novec (Registered Trademark) 7200 manufactured by 3M Japan Limited), and perfluorohexyl methyl ether (C₂F₅CF(OCH₃)C₃F₇) (e.g., Novec (Registered Trademark) 7300 manufactured by 3M Japan Limited), and CF₃CH₂OCF₂CHF₂ (e.g., Asahikrin (Registered Trademark) AE-3000 manufactured by AGC Inc.); and hydrofluoroolefins (HFO) (e.g., 1-chloro-2,3,3-trifluoro-1-propene (HCFO-1233 yd) (e.g., Amolea (Registered Trademark) AS-300 manufactured by AGC Inc.)).

The content of the liquid medium is preferably 60 to 99.999 wt. %, more preferably 80 to 99.99 wt. %, and still more preferably 90 to 99.9 wt. % based on the total amount of the composition according to the present disclosure. In the case where the composition according to the present disclosure is used in a wet coating method, the content of the liquid medium may be 90 to 99.99 wt. %, 95 to 99.98 wt. %, 97 to 99.97 wt. %, or 98 to 99.95 wt. % based on the total amount of the composition according to the present disclosure.

In addition to the compound according to the present disclosure and the liquid medium, the composition according to the present disclosure may contain other components in a range in which the effects of the present disclosure are not impaired. Examples of the other components include known additives such as acid catalysts and basic catalysts that accelerate the hydrolysis and the condensation reaction of the reactive silyl group.

As the catalyst, an arbitrary suitable acid or base, a transition metal (e.g., Ti, Ni, Sn, Zr, Al, or B), a sulfur-containing compound having a non-covalent electron pair in the molecular structure, a nitrogen-containing compound (e.g., a sulfoxide compound, an aliphatic amine compound, an aromatic amine compound, a phosphoric acid amide compound, an amide compound, and a urea compound) can be used.

Examples of acid catalysts include acetic acid, formic acid, trifluoroacetic acid, hydrochloric acid, nitric acid, sulfuric acid, phosphoric acid, sulfonic acid, methanesulfonic acid, and p-toluenesulfonic acid.

Further, examples of base catalysts include ammonia, sodium hydroxide, and potassium hydroxide; and organic amines such as triethylamine and diethylamine.

Further, examples of the other components also include metal compounds having hydrolyzable groups (hereinafter, metal compounds having hydrolyzable groups are also referred to as “specific metal compounds”). When the composition according to the present disclosure contains a specific metal compound, the sliding property and the antifouling property of the surface-treated layer can be further improved. Examples of specific metal compounds include those represented by the below-shown Formulas (M1) to (M3).

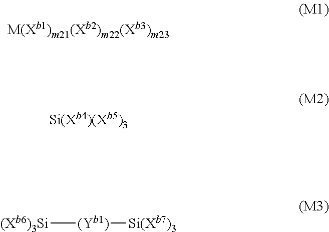

In Formula (M1),

M represents a trivalent or tetravalent metal atom.

X^{b1} each independently represents a hydrolyzable group.

X^{b2} each independently represents a siloxane skeleton-containing group.

X^{b3} each independently represents a hydrocarbon chain-containing group.
m21 is an integer of 2 to 4,
m22 and m23 each independently represent an integer of 0 to 2, and
when M is a trivalent metal atom, m21+m22+m23 is 3, whereas when M is a tetravalent metal atom, m21+m22+m23 is 4.

### In Formula (M2).

X^{b4} represents a hydrolyzable silane oligomer group.

X^{b5} each independently represents a hydrolyzable group or an alkyl group having a number of carbon atoms of 1 to 4.

In Formula (M3),

X^{b6} and X^{b7} each independently represent a hydrolyzable group or a hydroxyl group.

Y^{b1} represents a divalent organic group.

The metal represented by M in Formula (M1) includes semimetals such as Si and Ge. Mis preferably a trivalent metal and a tetravalent metal, more preferably Al, Fe, In, Hf, Si, Ti, Sn, and Zr, still more preferably Al, Si, Ti, and Zr, and particularly preferably Si.

Examples of the hydrolyzable group represented by X^{b1} in Formula (M1) include hydrolyzable groups similar to those represented by Lin Formula (G).

The siloxane skeleton-containing group represented by X^{b2} has a siloxane unit (—Si—O—) and may be a linear chain or a branched chain. The siloxane unit is preferably a dialkylsilyloxy group, and examples include a dimethylsilyloxy group and a diethylsilyloxy group. The number of repetitions of the siloxane unit in the siloxane skeleton-containing group is 1 or greater, preferably 1 to 5, more preferably 1 to 4, and still more preferably 1 to 3.

The siloxane skeleton-containing group may contain a divalent hydrocarbon group in a part of the siloxane skeleton. Specifically, an oxygen atom in a part of the siloxane skeleton may be replaced with a divalent hydrocarbon group. Examples of the aforementioned divalent hydrocarbon group include alkylene groups such as a methylene group, an ethylene group, a propylene group, and a butylene group.

A hydrolyzable group, a hydrocarbon group (preferably an alkyl group), or the like may be bonded to a silicon atom at the end of the siloxane skeleton-containing group.

The number of atoms of the siloxane skeleton-containing group is preferably 100 or smaller, more preferably 50 or smaller, and still more preferably 30 or smaller.

The number of elements is preferably 10 or greater. The siloxane skeleton-containing group is preferably a group represented by *—(O—Si(CH₃)₂)ₙCH₃, where n is an integer of 1 to 5 and * indicates a part bonded to an adjacent atom.

The hydrocarbon chain-containing group represented by X^{b3} may be a group consisting solely of a hydrocarbon chain or a group having an etheric oxygen atom between carbon atoms of a hydrocarbon chain.

The hydrocarbon chain may be a linear chain or a branched chain, and is preferably a linear chain. The hydrocarbon chain may be a saturated hydrocarbon chain or an unsaturated hydrocarbon chain, and is preferably a saturated hydrocarbon chain. The number of carbon atoms of the hydrocarbon chain-containing group is preferably 1 to 3, more preferably 1 or 2, and still more preferably 1. The hydrocarbon chain-containing group is preferably an alkyl group, and more preferably a methyl group, an ethyl group, or a propyl group.

m21 is preferably 3 or 4.

The compound represented by below-shown Formula (M1) is preferably compounds represented by the below-shown Formulas (M1-1) to (M1-5) in which Mis Si, and more preferably a compound represented by the below-shown Formula (M1-1). The compound represented by the below-shown Formula (M1-1) is preferably tetraethoxysilane, tetramethoxysilane, or triethoxymethylsilane.

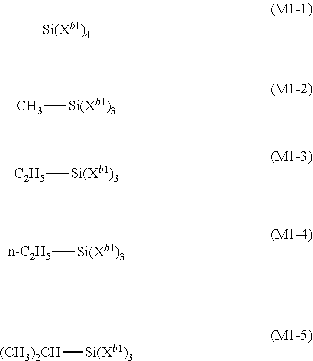

In Formula (M2), the number of silicon atoms contained in the hydrolyzable silane oligomer group represented by X^{b4} is preferably 3 or greater, more preferably 5 or greater, and still more preferably 7 or greater. The number of silicon atoms is preferably 15 or smaller, more preferably 13 or smaller, and still more preferably 10 or smaller.

The hydrolyzable silane oligomer group may have an alkoxy group that is bonded to a silicon atom. Examples of the alkoxy group include a methoxy group, an ethoxy group, a propoxy group, and a butoxy group. Further, the alkoxy group is preferably a methoxy group or an ethoxy group. The hydrolyzable silane oligomer group may have one or two or more groups of alkoxy groups, and preferably has one type of alkoxy group.

Examples of the hydrolyzable silane oligomer group include (C₂H₅O)₃Si—(OSi(OC₂H₅)₂)₄O—*. Note that * indicates a part bonded to an adjacent atom.

Examples of the hydrolyzable group represented by X^{b5} in Formula (M2) include hydrolyzable groups similar to those represented by L in the above-shown Formula (G), a cyano group, a hydrogen atom, and an allyl group. Further, the hydrolyzable group is preferably an alkoxy group or an isocyanato group. The alkoxy group is preferably an alkoxy group having a number of carbon atoms of 1 to 4.

X^{b5} is preferably a hydrolyzable group.

Examples of the compound represented by Formula (M2) include (H₅C₂O)₃—Si—(OSi(OC₂H₅)₂)₄OC₂H₅.

The compound represented by Formula (M3) is a compound having reactive silyl groups at both ends of a divalent organic group, i.e., is bissilane.

Examples of the hydrolyzable groups represented by X^{b6} and X^{b7} in Formula (M3) include an alkoxy group, an acyloxy group, a ketoxime group, an alkenyloxy group, an amino group, an aminoxy group, an amide group, an isocyanato group, and a halogen atom. Further, the hydrolyzable groups are preferably alkoxy groups or isocyanato groups. The alkoxy group is preferably an alkoxy group having a number of carbon atoms of 1 to 4, and more preferably a methoxy group or an ethoxy group.

In Formula (M3), X^{b6} and X^{b7} may be the same groups as each other or groups different from each other. In view of the availability, X^{b6} and X^{b7} are preferably the same groups as each other.

In Formula (M3), Y^{b1} is a divalent organic group connecting reactive silyl groups at both ends. The number of carbon atoms of Y^{b1} of the divalent organic group is preferably 1 to 8 and more preferably 1 to 3.

Examples of Y^{b1} include an alkylene group, a phenylene group, and an alkylene group having an etheric oxygen atom between carbon atoms. Examples include —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂C(CH₃)₂CH₂—, —C(CH₃)₂CH₂CH₂C(CH₃)₂—, —CH₂CH₂OCH₂CH₂—, —CH₂CH₂CH₂OCH₂CH₂CH₂—, —CH(CH₃) CH₂OCH₂CH(CH₃)—, and —C₆H₄—.

Examples of the compound represented by Formula (M3) include (CH₃O)₃Si(CH₂)₂Si(OCH₃)₃, (C₂H₅O)₃Si(CH₂)₂Si(OC₂H₅)₃, (OCN)₃Si(CH₂)₂Si(NCO)₃, Cl₃Si(CH₂)₂SiCl₃, (CH₃O)₃Si(CH₂)₆Si(OCH₃)₃, and (C₂H₅O)₃Si(CH₂)₆Si(OC₂H₅)₃.

The content of other components which may be contained in the composition according to the present disclosure is preferably 10 mass % or less and more preferably 1 mass % or less based on the total amount of the composition according to the present disclosure. When the composition according to the present disclosure contains a specific metal compound, the content of the specific metal compound is preferably 0.01 to 30 mass %, more preferably 0.01 to 10 mass %, and still more preferably 0.05 to 5 mass % based on the total amount of the composition according to the present disclosure.

The total content (hereinafter also referred to as “solid content concentration”) of the compound according to the present disclosure and the other components is preferably 0.001 to 40 wt. %, more preferably 0.01 to 20 wt. %, and still more preferably 0.1 to 10 wt. % based on the total amount of the composition according to the present disclosure. The solid content concentration of the composition according to the present disclosure is a value calculated from the mass of the composition before being heated and the mass thereof after being heated in a convection-type dryer at 120° C. for 4 hours.

Since the composition according to the present disclosure contains a liquid medium, it is useful for use in which the composition is used for coating, and can be used as a coating liquid.

Examples of the other components include compounds represented by below-shown Formula (3).

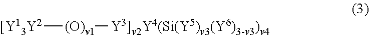

In Formula (1), Y1 is each independently a hydrocarbon group or a trialkylsilyloxy group,
Y² is Si, Sn, or Ge,
v1 is 0 or 1,
Y³ is each independently an alkylene chain or a polyalkylene oxide chain, or a combination of an alkylene chain and a divalent polysiloxane residue,
Y⁴ is a single bond or a linking group having a valence of (v2+v4),
Y⁵ is each independently a hydrocarbon group,
Y⁶ is each independently a hydrolyzable group or a hydroxyl group,
v3 is each independently an integer of 0 to 2, and
v2 and v4 are each independently an integer of 1 or greater.

The compound (3) is preferably a compound in which Y³ is an alkylene chain or a polyalkylene oxide chain.

Specific examples of the compound (3) include compounds shown below. γ in the formula is preferably 9 to 50, more preferably 11 to 30, and particularly preferably 11 or 25.

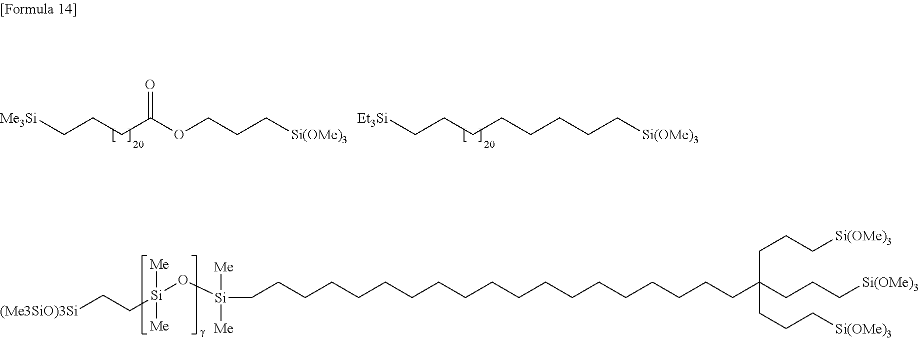

When the other component in the surface treatment agent disclosed herein is the compound (3), the content of the compound (3) is preferably 50 mass % or less and more preferably 40 mass % or less.

### [Surface Treatment Agent]

In an embodiment, a surface treatment agent according to the present disclosure contains a compound according to the present disclosure. Further, a surface treatment agent according to the present disclosure may contain a compound according to the present disclosure and a liquid medium. A surface treatment agent according to the present disclosure may be a composition according to the present disclosure. Preferred embodiments of the liquid medium contained in the surface treatment agent according to the present disclosure are similar to those of the liquid medium contained in the composition according to the present disclosure.

The compound according to the present disclosure contains an alkyl group having a number of carbon atoms of 2 or greater, an organopolysiloxane group, and a reactive silyl group. Therefore, by using the surface treatment agent comprising the compound according to the present disclosure, a surface-treated layer having excellent water repellency and excellent abrasion resistance can be formed.

In particular, the surface treatment agent according to the present disclosure is preferably used for an optical member.

### [Article]

In an embodiment, an article according to the present disclosure includes a substrate and a surface-treated layer which is disposed on the substrate and of which the surface is treated with a surface treatment agent according to the present disclosure.

The surface-treated layer may be formed on a part of the surface of the substrate or over the entire surface of the substrate. The surface-treated layer may be spread in the form of a film on the surface of the substrate or scattered in the form of dots.

In the surface-treated layer, the compound according to the present disclosure is contained therein in a state in which the hydrolysis of some or all of reactive silyl groups have progressed and the dehydration condensation reaction of silanol groups have progressed.

The thickness of the surface-treated layer is preferably 1 to 100 nm and more preferably 1 to 50 nm. When the thickness of the surface-treated layer is 1 nm or larger, the effects of the surface treatment are likely to be obtained satisfactorily. When the thickness of the surface-treated layer is 100 nm or smaller, the usage efficiency is high. The thickness of the surface-treated layer can be calculated from the oscillation cycle of the interference pattern of the reflected X-ray, which is obtained by an X-ray reflectivity method using an X-ray diffractometer for thin film analysis (Product name: ATX-G, manufactured by Rigaku Corporation).

The type of the substrate is not limited to any particular types, and examples include a substrate which is required to have water repellency. Examples of the substrate include a substrate which may be used while touching it with another article (e.g., a stylus) or a human hand or fingers; a substrate which may be held by a human hand or fingers during the operation; and a substrate which may be placed on another article (e.g., a mounting table).

Examples of the material of the substrate include metals, resins, glass, sapphire, ceramics, semiconductors, stones, fibers, nonwoven fabric, paper, wood, fur, natural leather, artificial leather, ceramics, and composite materials thereof. The glass may be one that is chemically reinforced.

Examples of substrates including building materials, decorative building materials, interior goods, transportation apparatuses (e.g., automobiles), signboards, bulletin boards, drinking containers, tableware, water tanks, ornamental apparatuses (e.g., frames and boxes), laboratory apparatuses, furniture, textile products, and packaging containers; glass or resins used for art, sports, games and the like; glass or resins used for exterior parts (excluding display parts) of apparatuses such as mobile phones (e.g., smartphones), mobile information terminals, game machines, and remote controllers. The shape of the substrate may be plate-like or film-like.

The substrate is preferably a substrate for a touch panel, a substrate for a display, or a lens for eyeglasses, and particularly preferably a substrate for a touch panel. The material of a substrate for a touch panel is preferably glass or a transparent resin.

The substrate may be a substrate of which a surface treatment such as a corona discharge treatment, a plasma treatment, a plasma graft polymerization treatment, or the like has been performed on one or both of the surfaces. The substrate subjected to the surface treatment has a more excellent adhesive property for the surface-treated layer, and the abrasion resistance of the surface-treated layer is further improved. Therefore, it is preferred to perform a surface treatment on the surface of the substrate on the side which is brought into contact with the surface-treated layer. Further, in the case where an underlayer (which will be described later) is provided, the substrate subjected to the surface treatment has a more excellent adhesive property for the underlayer, and the abrasion resistance of the surface-treated layer is further improved. Therefore, in the case where an underlayer is provided, it is preferred to perform a surface treatment on the surface of the substrate on the side which is brought into contact with the underlayer.

The surface-treated layer may be directly disposed on the surface of the substrate, or an underlayer may be provided between the substrate and the surface-treated layer. In order to further improve the water repellency and the abrasion resistance of the surface-treated layer, an article according to the present disclosure preferably includes a substrate, an underlayer disposed on the substrate, and a surface-treated layer which is disposed on the underlayer and of which the surface is treated with a surface treatment agent according to the present disclosure.

The underlayer is preferably a layer comprising an oxide comprising silicon and at least one specific element selected from the group consisting of Group 1 elements, Group 2 elements, Group 4 elements, Group 5 elements, Group 13 elements, and Group 15 elements in the periodic table.

Group 1 elements in the periodic table (hereinafter also referred to simply as “Group 1 elements”) mean lithium, sodium, potassium, rubidium, and cesium. Group 1 elements are preferably lithium, sodium, and potassium, and more preferably sodium and potassium because the surface-treated layer can be formed more uniformly on the underlayer without defects or because variations in the composition of the underlayer among samples are more suppressed. The underlayer may contain two or more Group 1 elements.

Group 2 elements in the periodic table (hereinafter also referred to simply as “Group 2 elements”) mean beryllium, magnesium, calcium, strontium, and barium. Group 2 elements are preferably magnesium, calcium, and barium, and more preferably magnesium and calcium because the surface-treated layer can be formed more uniformly on the underlayer without defects or because variations in the composition of the underlayer among samples are more suppressed. The underlayer may contain two or more Group 2 elements.

Group 4 elements in the periodic table (hereinafter also referred to simply as “Group 4 elements”) mean titanium, zirconium, and hafnium. Group 4 elements are preferably titanium and zirconium, and more preferably titanium because the surface-treated layer can be formed more uniformly on the underlayer without defects or because variations in the composition of the underlayer among samples are more suppressed. The underlayer may contain two or more Group 4 elements.

Group 5 elements in the periodic table (hereinafter also referred to simply as “Group 5 elements”) mean vanadium, niobium, and tantalum. Group 5 elements are particularly preferably vanadium because the abrasion resistance of the surface-treated layer becomes more excellent. The underlayer may contain two or more Group 5 elements.

Group 13 elements in the periodic table (hereinafter also referred to simply as “Group 13 elements”) mean boron, aluminum, gallium, and indium. Group 13 elements are preferably boron, aluminum, and gallium, and more preferably boron and aluminum because the surface-treated layer can be formed more uniformly on the underlayer without defects or because variations in the composition of the underlayer among samples are more suppressed. The underlayer may contain two or more Group 13 elements.

Group 15 elements in the periodic table (hereinafter also referred to simply as “Group 15 elements”) mean nitrogen, phosphorus, arsenic, antimony, and bismuth. Group 15 elements are preferably phosphorus, antimony, and bismuth, and more preferably phosphorus and bismuth because the surface-treated layer can be formed more uniformly on the underlayer without defects or because variations in the composition of the underlayer among samples are more suppressed. The underlayer may contain two or more Group 15 elements.

The specific element contained in the underlayer is preferably a Group 1 element, a Group 2 element, and a Group 13 element, more preferably a Group 1 element and a Group 2 element, and still more preferably a Group 1 element because the abrasion resistance of the surface-treated layer becomes more excellent.

Only one element or two or more elements may be contained as the specific element(s).

The oxide contained in the underlayer may be a mixture of oxides each comprising only one of the aforementioned elements (silicon and specific element) (e.g., a mixture of silicon oxide and an oxide of a specific element), a composite oxide comprising two or more of the aforementioned elements, or a mixture of an oxide comprising only one of the aforementioned elements and a composite oxide.

The ratio of the total molarity of the specific element in the underlayer to the molarity of silicon in the underlayer (specific element/silicon) is preferably 0.02 to 2.90, more preferably 0.10 to 2.00, and still more preferably 0.20 to 1.80 because the abrasion resistance of the surface-treated layer becomes more excellent.

The molarity (mol %) of each element in the underlayer can be measured, for example, by a depth-direction analysis by X-ray photoelectron spectroscopy (XPS) using ion sputtering.

The underlayer may be a single layer or may consist of a plurality of layer.

The underlayer may have an uneven surface(s). The thickness of the underlayer is preferably 1 to 100 nm, more preferably 1 to 50 nm, and still more preferably 2 to 20 nm. When the thickness of the underlayer is equal to or larger than the aforementioned lower limit value, the adhesive property of the surface-treated layer by the underlayer is further improved, so that the abrasion resistance of the surface-treated layer becomes more excellent. When the thickness of the underlayer is equal to or smaller than the aforementioned upper limit value, the abrasion resistance of the underlayer itself becomes excellent.

The thickness of the underlayer is measured by observing the cross section of the underlayer by a transmission electron microscope (TEM).

The underlayer can be formed, for example, by a vapor-deposition method using a vapor-deposition material or a wet coating method.

The vapor-deposition material used in the vapor-deposition method preferably an oxide comprising silicon and a specific element.

Specific examples of the form of the vapor-deposition material include a powder, a molten material, a sintered compact, granules, and a crushed material. Further, a molten material, a sintered compact, and granules are preferred in view of the handling property.

Note that the molten material means a solid material obtained by melting a powder of a vapor-deposition material at a high temperature, and then cooling and solidifying the molten vapor-deposition material. The sintered compact means a solid material obtained by firing a powder of a vapor-deposition material. Further, if necessary, instead of the powder of the vapor-deposition material, a molded compact formed by press-molding such a powder may be used. The granules means a solid material obtained by mixing and kneading a powder of a vapor-deposition material and a liquid medium (e.g., water or an organic solvent) and thereby obtaining particles thereof, and then drying the obtained particles.

The vapor-deposition material can be manufactured, for example, by the following methods.
A method in which a powder of a vapor-deposition material is obtained by mixing a powder of silicon oxide with a powder of an oxide of a specific element.
A method in which granules of a vapor-deposition material is obtained by, after obtaining particles by mixing and kneading a powder of the aforementioned vapor-deposition material and water, drying the obtained particles.
A method in which a sintered compact is obtained by drying a mixture obtained by mixing a powder comprising silicon (e.g., a powder made of silicon oxide, silica sand, or silica gel), a powder comprising a specific element (e.g., a powder of an oxide of a specific element, a carbonate, a sulfate, a nitrate, an oxalate, or a hydroxide), and water, and then firing the dried mixture or a molded article obtained by press-molding the dried mixture.
A method in which a molten material is obtained by melting a mixture obtained by mixing a powder comprising silicon (e.g., a powder made of silicon oxide, silica sand, or silica gel), a powder comprising a specific element (e.g., a powder of an oxide of a specific element, a carbonate, a sulfate, a nitrate, an oxalate, or a hydroxide), and water at a high temperature, and then cooling and solidifying the molten substance.

Specific examples of the vapor-deposition method using a vapor-deposition material include a vacuum vapor-deposition method. The vacuum vapor-deposition method is a method in which a vapor-deposition material is evaporated in a vacuum chamber and deposited on the surface of a substrate.

The temperature during the vapor-deposition (e.g., in the case where a vacuum deposition apparatus is used, the temperature of a boat in which the deposition material is disposed) is preferably 100 to 3,000° C. and more preferably 500 to 3,000° C.

The pressure during the vapor-deposition (e.g., in the case where a vacuum deposition apparatus is used, the pressure in a chamber in which the deposition material is disposed) is preferably 1 Pa or lower and more preferably 0.1 Pa or lower.

When an underlayer is formed by using a vapor-deposition material, one vapor-deposition material may be used, or two or more vapor-deposition materials comprising different elements may be used.

Specific examples of the method for evaporating a vapor-deposition material include a resistive heating method in which a vapor-deposition material is melted and evaporated on a resistive heating boat made of a high melting-point metal, and an electron gun method in which a vapor-deposition material is irradiated with an electron beam and thereby directly heated, so that its surface is melted and evaporated. As the method for evaporating a vapor-deposition material, the electron gun method is preferred because since the material can be locally heated, even a material having a high melting point can be evaporated, and since an area that is not irradiated with the electron beam is kept at a low temperature, there is no risk of reaction with the container nor risk of contamination by impurities.

As the method for evaporating a vapor-deposition material, a plurality of boats may be used, or the whole vapor-deposition materials may be put in a single boat. The vapor-deposition method may be co-vapor deposition or alternating vapor deposition. Specific examples include an example in which silica and a specific element source are mixed and used in the same boat, an example in which co-vapor deposition is performed in a state in which silica and a specific element source put in separate boats, and an example in which alternating vapor deposition is performed in a similar manner, i.e., in a state in which silica and a specific element source are put in separate boats. The conditions for the vapor deposition, the order, and the like are selected as appropriate according to the structure of the underlayer.

In the wet coating method, it is preferred to form an underlayer on a substrate by a wet coating method using a coating liquid comprising a compound comprising silicon, a compound comprising a specific element, and a liquid medium.

Specific examples of silicon compounds include silicon oxide, silicic acid, partial condensate of silicic acid, alkoxysilane, and partial hydrolysis condensate of alkoxysilane.

Specific examples of compounds comprising a specific element include oxides of the specific element, alkoxides of the specific element, carbonates of the specific element, sulfates of the specific element, nitrates of the specific element, oxalates of the specific element, and hydroxides of the specific element.

Examples of liquid mediums include those similar to liquid mediums contained in the composition according to the present disclosure.

The content of the liquid medium is preferably 0.01 to 20 mass % and more preferably 0.1 to 10 mass % based on the total amount of the coating liquid used for the formation of the underlayer.

Specific examples of wet coating methods for forming an underlayer include a spin coating method, a wipe coating method, a spray coating method, a squeegee coating method, a dip coating method, a die coating method, an ink jet method, a flow coating method, a roll coating method, a casting method, a Langmuir-Blodgett method, and a gravure coating method.

After the substrate or the like is wet-coated with the coating liquid, the coating is preferably dried. The drying temperature of the coating is preferably 20 to 200° C. and more preferably 80 to 160° C.

The article according to the present disclosure may be an optical material including a surface-treated layer as the outermost layer.

Examples of preferred optical materials include a wide variety of optical materials in addition to optical materials related to displays or the like.

Examples of optical materials include a cathode ray tube (CRT; e.g., a computer monitor), a display such as a liquid crystal display, a plasma display, an organic EL display, an inorganic thin film EL dot matrix display, a rear projection-type display, a vacuum fluorescent display (VFD), and a field emission display (FED), a protective plate for such a display, and those in which an antireflection film treatment is performed on their surfaces.

The article according to the present disclosure is preferably an optical member. Examples of optical members include a car navigation system, a mobile phone, a smartphone, a digital camera, a digital video camera, a PDA, a portable audio player, a car audio system, a game apparatus, a lens for eyeglasses, a camera lens, a lens filter, sunglasses, a medical apparatus such as a gastroscope, a copying machine, a PC, a display (e.g., a liquid crystal display, an organic EL display, a plasma display, and a touch panel display), a touch panel, a protective film, and an antireflection film.

Further, examples of optical members include a front protective plate for a display such as a PDP and an LCD, an antireflection plate, a polarizing plate, and an antiglare plate; a disk surface of an optical disk such as a Blu-ray (Blu-ray (Registered Trademark)) disk, a DVD disk, a CD-R, and an MO; an optical fiber; and a display surface of a clock or a watch.

In particular, the article according to the present disclosure is preferably a display or a touch panel.

The article according to the present disclosure may be a medical apparatus or a medical material. Further, the article according to the present disclosure may also be an automobile interior or exterior member. Examples of exterior members include a window, a light cover, and an external camera cover. Examples of interior members include an instrument panel cover, a navigation system touch panel, and a decorative interior member.

When the article according to the present disclosure is an optical member, the material constituting the surface of the substrate is a member for an optical member, e.g., glass or transparent plastic. Further, when the article according to the present disclosure is an optical member, a functional layer such as a hard coat layer or an antireflection layer may be formed on the surface (outermost layer) of the substrate. The antireflection layer may be either a single-layer antireflection layer or a multi-layer antireflection layer.

Examples of inorganic substances that can be used for the antireflection layer include SiO₂, SiO, ZrO₂, TiO₂, TiO, Ti₂O₃, Ti₂O₅, Al₂O₃, Ta₂O₅, Ta₃O₅, Nb₂O₅, HfO₂, Si₃N₄, CeO₂, MgO, Y₂O₃, SnO₂, MgF₂, and WO₃. These inorganic substances may be used alone or in combination (e.g., as a mixture) of two or more of them. In the case of the multi-layer antireflection layer, it is preferred to use SiO₂ and/or SiO in the outermost layer. When the article according to the present disclosure is an optical glass component for a touch panel, a thin film using a transparent electrode, e.g., indium tin oxide (ITO), indium zinc oxide, or the like, may be provided on a part of the surface of the substrate (glass). Further, the substrate may also include an insulating layer, an adhesive layer, a protective layer, a decorative frame layer (I-CON), an atomization film layer, a hard coating film layer, a polarizing film, a phase difference film, a liquid crystal display module, or the like according to its specific specifications.

### [Method for Manufacturing Article]

A method for manufacturing an article according to the present disclosure is, for example, a method for manufacturing an article including a surface-treated layer formed on a substrate by performing a surface treatment on the substrate by using a surface treatment agent according to the present disclosure. Examples of surface treatments include a dry coating method and a wet coating method.

Examples of dry coating methods include techniques such as vacuum vapor deposition, CVD, and sputtering. As the dry coating method, a vacuum vapor-deposition method is preferred in order to suppress the decomposition of the compound and in view of the simplicity of the apparatus. During the vacuum deposition, a pellet-like substance obtained by impregnating a porous material made of a metal such as iron or steel with a compound according to the present disclosure may be used. A pellet-like substance, which is obtained by impregnating a porous material made of a metal such as iron or steel with a composition comprising a compound according to the present disclosure and a liquid medium, and drying the liquid medium, may be used.

Examples of wet coating methods include a spin coating method, a wipe coating method, a spray coating method, a squeegee coating method, a dip coating method, a die coating method, an ink jet method, a flow coating method, a roll coating method, a casting method, a Langmuir-Blodgett method, and a gravure coating method.

In order to improve the abrasion resistance of the surface-treated layer, when necessary, an operation for accelerating the reaction between the compound according to the present disclosure and the substrate may be performed.

Examples of such operations include heating, humidification, and irradiation with light. For example, it is possible to accelerate, by heating a substrate on which a surface-treated layer is formed in an atmosphere comprising moisture, the reaction such as a hydrolysis reaction of a hydrolyzable group, a reaction between a hydroxyl group or the like and a silanol group on the surface of the substrate, and formation of a siloxane bond by a condensation reaction of a silanol group.

After the surface treatment, compounds which are contained in the surface-treated layer and are not chemically bonded to other compounds nor the substrate may be removed as required. Examples of removal methods include a method in which a solvent is poured over the surface-treated layer, and a method in which the surface-treated layer is wiped with a cloth impregnated with a solvent.

### EXAMPLES

The present invention will be described hereinafter in a more detailed manner by using examples, but the present invention is not limited to these examples. Note that Examples 1 to 6 are examples according to the present disclosure, and Examples 7 and 8 are comparative examples.

### [Compound 1-3]

### <Synthesis of Compound 1-1>

THF (Tetrahydrofuran, 101 g) was added to hexamethylcyclotrisiloxane (76 g), and the mixture was stirred at 25° C. until the hexamethylcyclotrisiloxane was dissolved. Next, a solution in which lithium salt of trimethylsilanol (5.1 g) was suspended in THF (20 g) was added to the resultant solution, and the mixture was stirred at 25° C. for 2 hours. Next, a reaction solution was obtained by adding chlorodimethylsilane (10.5 g) and stirring the mixture at 25° C. for 1 hour. Extraction was performed by adding hexane and water to the reaction solution. Then, 25 g of a compound 1-1 was obtained by removing the solvent and low-boiling components by distillation under a reduced pressure and performing flash column chromatography (developing solvent: hexane/dichloromethane) using silica gel. The average value of n in the compound 1-1 was 17. The structure of the compound 1-1 was confirmed from NMR data shown below.

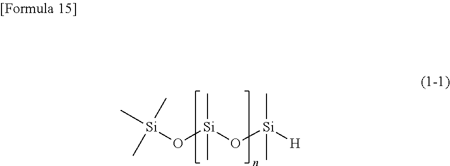

¹H-NMR (400 MHz, CDCl₃) δ:4.63 (hept, J=2.8 Hz, 1H), 0.21-0.08 (m, 6H), 0.07-−0.12 (m, 111H).

### <Synthesis of Compound 1-2>

Dichloromethane (100 g) and trichloroisocyanuric acid (14 g) were added to the compound 1-1 (10 g), and the mixture was stirred at 25° C. for 3 hours. Next, after removing insoluble substances by filtering the resultant solution, low-boiling components were removed by distilling the filtrate under a reduced pressure. A reaction solution was obtained by adding methanol (30 g) to the obtained crude solution and stirring the mixture at 60° C. for 2 hours. Extraction was performed by adding hexane and water to the reaction solution. Then, 5.2 g of a compound 1-2 was obtained by removing the solvent and low-boiling components by distillation under a reduced pressure and performing flash column chromatography (developing solvent: hexane/ethyl acetate) using silica gel. The average value of n in the compound 1-2 was 17. The structure of the compound 1-2 was confirmed from NMR data shown below.]

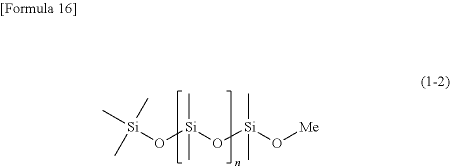

¹H-NMR (400 MHz, CDCl₃) δ: 3.46 (s, 3H), 0.32-−0.05 (m, 117H).

### <Synthesis of Compound 1-3>

The compound 1-2 (1 g) and toluene (10 g) were added to 2,4,6,8-tetramethylcyclotetrasiloxane (10 g), and the mixture was stirred at 25° C. until it became homogeneous. Next, tris (pentafluorophenyl) borane (0.06 g) was added, and the mixture was stirred at 25° C. for 2 hours. After removing the solvent and unreacted 2,4,6,8-tetramethylcyclotetrasiloxane by distillation under a reduced pressure, tetramethyl orthosilicate (3.0 g) and toluene (10 g) were added, and the mixture was stirred at 25° C. until it became homogeneous. Next, tris (pentafluorophenyl) borane (0.06 g) was added, and the mixture was stirred at 25° C. for 2 hours. 1.5 g of a compound 1-3 was obtained by removing the solvent and unreacted tetramethyl orthosilicate by distillation under a reduced pressure. The average value of n in the compound 1-3 was 17. The structure of the compound 1-3 was confirmed from NMR data shown below.

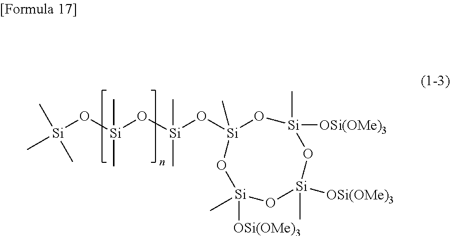

¹H-NMR (400 MHz, CDCl₃) δ: 3.76-3.26 (m, 27H), 0.32-−0.15 (m, 129H).

### [Compound 2-1]

The compound 1-2 (1 g) and toluene (10 g) were added to octakis (dimethylsilyloxy) octasylsesquioxane (20 g), and the mixture was stirred at 25° C. until it became homogeneous. Next, tris (pentafluorophenyl) borane (0.06 g) was added, and the mixture was stirred at 25° C. for 2 hours. Unreacted octakis (dimethylsilyloxy) octasylsesquioxane was removed by performing, for the obtained crude solution, flash column chromatography (developing solvent: hexane/dichloromethane) using silica gel. Next, tetramethyl orthosilicate (20 g) and toluene (10 g) were added, and the mixture was stirred at 25° C. until it became homogeneous. Then, tris (pentafluorophenyl) borane (0.06 g) was added, and the mixture was stirred at 25° C. for 2 hours. 0.6 g of a compound 2-1 was obtained by removing the solvent and unreacted tetramethyl orthosilicate by distillation under a reduced pressure. The average value of n in the compound 2-1 was 17. The structure of the compound 2-1 was confirmed from NMR data shown below.

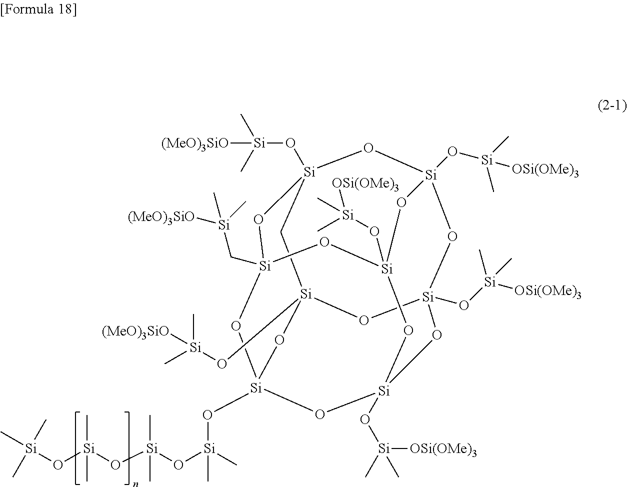

¹H-NMR (400 MHz, CDCl₃) δ:3.82-3.22 (m, 63H), 0.41-−0.15 (m, 165H).

### [Compound 3-1]

The compound 1-2 (1 g) and toluene (10 g) were added to tetrakis(dimethylsilyloxy) silane (20 g), and the mixture was stirred at 25° C. until it became homogeneous. Next, tris (pentafluorophenyl) borane (0.06 g) was added, and the mixture was stirred at 25° C. for 2 hours. The solvent and unreacted tetrakis(dimethylsilyloxy) silane were removed by distillation under a reduced pressure. Next, tetramethyl orthosilicate (10 g) and toluene (10 g) were added, and the mixture was stirred at 25° C. until it became homogeneous. Then, tris (pentafluorophenyl) borane (0.06 g) was added, and the mixture was stirred at 25° C. for 2 hours. 1.5 g of a compound 3-1 was obtained by removing the solvent and unreacted tetramethyl orthosilicate by distillation under a reduced pressure. The average value of n in the compound 3-1 was 17. The structure of the compound 3-1 was confirmed from NMR data shown below.

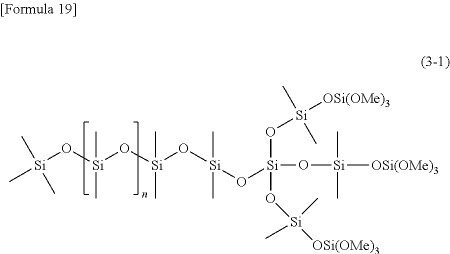

¹H-NMR (400 MHz, CDCl₃) δ: 3.64-3.35 (m, 27H), 0.31-−0.13 (m, 141H).

### [Formula 4-1]

SILANOL TERMINATED POLYDIMETHYLSILOXANE (molecular weight: about 1,000 g/mol) (1.0 g) and toluene (10 g) were added to tetrakis(dimethylsilyloxy) silane (20 g), and the mixture was stirred at 25° C. until it became homogeneous. Next, tris (pentafluorophenyl) borane (0.06 g) was added, and the mixture was stirred at 25° C. for 2 hours. The solvent and unreacted tetrakis (dimethylsilyloxy) silane were removed by distillation under a reduced pressure. Next, tetramethyl orthosilicate (10 g) and toluene (10 g) were added, and the mixture was stirred at 25° C. until it became homogeneous. Then, tris (pentafluorophenyl) borane (0.06 g) was added, and the mixture was stirred at 25° C. for 2 hours. 1.7 g of a compound 4-1 was obtained by removing the solvent and unreacted tetramethyl orthosilicate by distillation under a reduced pressure. The average value of n in the compound 4-1 was 14. The structure of the compound 4-1 was confirmed from NMR data shown below.

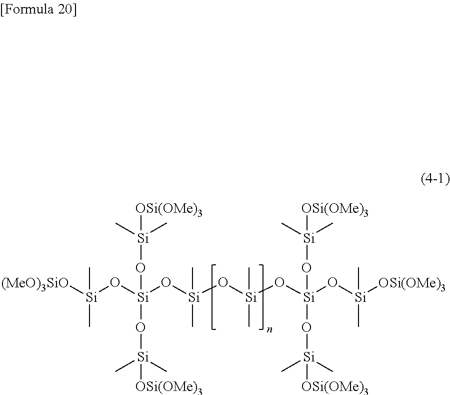

¹H-NMR (400 MHz, CDCl₃) δ: 3.64-3.35 (m, 54H), 0.31-−0.13 (m, 126H).

### [Compound 5-1]

The compound 1-2 (1 g) and toluene (10 g) were added to 3-[(dimethylsilyl)oxy]-1,1,3,5,5-pentamethyltrisiloxane (20 g), and the mixture was stirred at 25° C. until it became homogeneous. Next, tris (pentafluorophenyl) borane (0.06 g) was added, and the mixture was stirred at 25° C. for 2 hours. The solvent and unreacted tetrakis (dimethylsilyloxy) silane were removed by distillation under a reduced pressure. Next, tetramethyl orthosilicate (10 g) and toluene (10 g) were added, and the mixture was stirred at 25° C. until it became homogeneous. Then, tris (pentafluorophenyl) borane (0.06 g) was added, and the mixture was stirred at 25° C. for 2 hours. 1.5 g of a compound 5-1 was obtained by removing the solvent and unreacted tetramethyl orthosilicate by distillation under a reduced pressure. The average value of n in the compound 5-1 was 17. The structure of the compound 5-1 was confirmed from NMR data shown below.

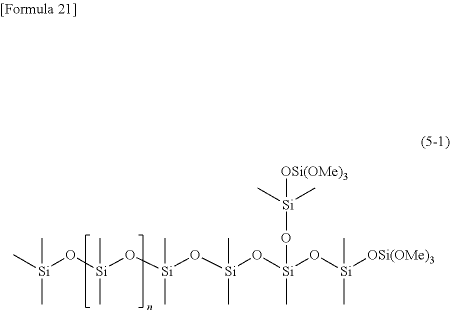

¹H-NMR (400 MHz, CDCl₃) δ: 3.64-3.35 (m, 18H), 0.31-−0.13 (m, 138H).

### [Compound 6-3]

### <Synthesis of Compound 6-1>

Dichloromethane (100 g) and trichloroisocyanuric acid (14 g) were added to 1,1,1,3,5,5,5-heptamethyltrisiloxane (10 g), and the mixture was stirred at 25° C. for 3 hours. Next, after removing insoluble substances by filtering the reaction solution, low-boiling components were removed by distilling the filtrate. Water (20 g), THF (40 g), and triethylamine (10 g) were added to the obtained crude solution, and the mixture was stirred at 25° C. for 2 hours. The extraction was performed by adding hexane and water, and low-boiling components were removed by distillation. 8.3 g of a compound 6-1 was obtained by performing, for the obtained crude solution, flash column chromatography (developing solvent: hexane/ethyl acetate) using silica gel. The structure of the compound 6-1 was confirmed from NMR data shown below.

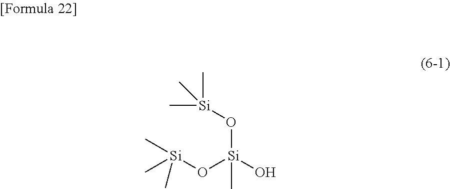

¹H-NMR (400 MHz, CDCl₃) δ:2.41 (q, J=7.2 Hz, 1H), 0.20-0.21 (m, 21H).

### <Synthesis of Compound 6-2>

THF (20 g) was added to the compound 6-1 (2.0 g), and the mixture was cooled to 0° C. Then, a methyllithium solution (3.1M in diethoxymethane) (2.7 mL) was added, and the mixture was stirred at 25° C. for 10 minutes. Next, a solution in which hexamethylcyclotrisiloxane (5.6 g) was dissolved in THF (20 g) was added to the resultant solution, and the mixture was stirred at 25° C. for 4 hours. After performing extraction by adding hexane and hydrochloric acid, low-boiling components were removed by distillation. 4.3 g of a compound 6-2 was obtained by performing, for the obtained crude solution, flash column chromatography (developing solvent: hexane/dichloromethane) using silica gel. The average value of n in the compound 6-2 was 8. The structure of the compound 6-2 was confirmed from NMR data shown below.

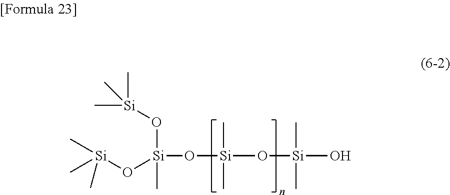

¹H-NMR (400 MHz, CDCl₃) δ:0.41-−0.15 (m, 75H).

### <Synthesis of Compound 6-3>

The compound 6-2 (1.0 g) and toluene (10 g) were added to tetrakis (dimethylsilyloxy) silane (20 g), and the mixture was stirred at 25° C. until it became homogeneous. Next, tris (pentafluorophenyl) borane (0.06 g) was added, and the mixture was stirred at 25° C. for 2 hours. The solvent and unreacted tetrakis (dimethylsilyloxy) silane were removed by distillation under a reduced pressure. Next, tetramethyl orthosilicate (10 g) and toluene (10 g) were added, and the mixture was stirred at 25° C. until it became homogeneous. Then, tris (pentafluorophenyl) borane (0.06 g) was added, and the mixture was stirred at 25° C. for 2 hours. 1.7 g of a compound 6-3 was obtained by removing the solvent and unreacted tetramethyl orthosilicate by distillation under a reduced pressure. The average value of n in the compound 6-3 was 8. The structure of the compound 6-3 was confirmed from NMR data shown below.

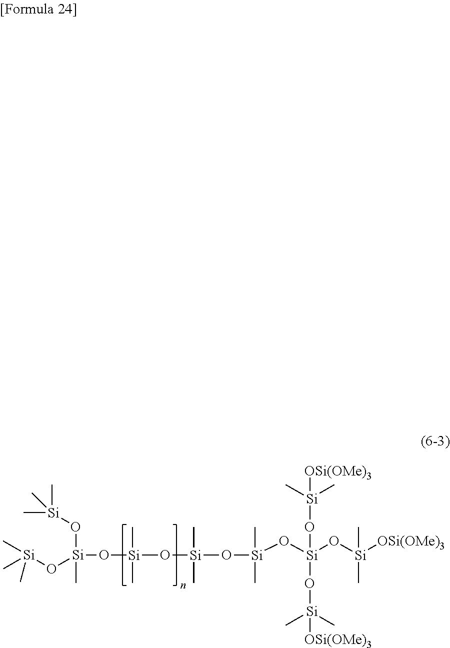

¹H-NMR (400 MHz, CDCl₃) δ: 3.64-3.35 (m, 27H), 0.31-−0.13 (m, 99H).

### [Synthesis of Compound 7-1]

A compound 7-1 was obtained according to a method described in Japanese Unexamined Patent Application Publication No. 2017-119849. The average value of n in the compound 7-1 was 17.

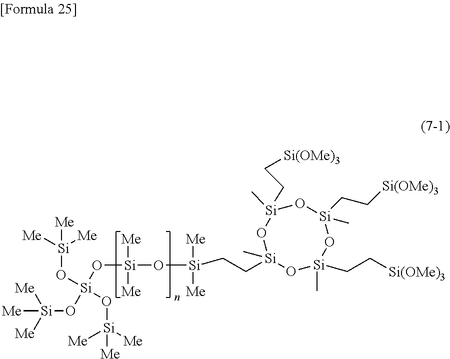

### [Synthesis of Compound 8-1]

A compound 8-1 was obtained according to a method described in Japanese Unexamined Patent Application Publication No. 2017-119849. The average value of n in the compound 8-1 was 17.

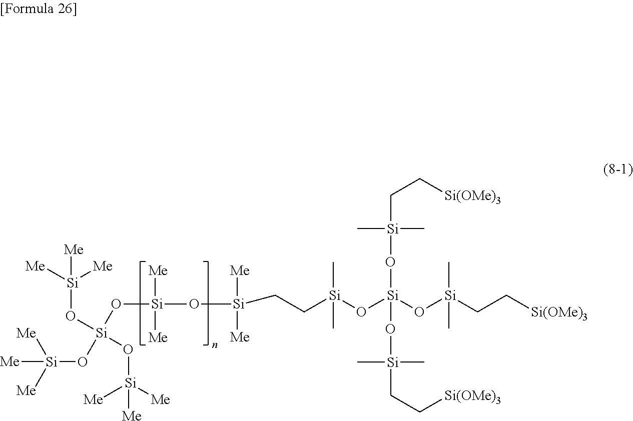

### [Manufacturing of Article]

Articles in Examples 1 to 5 were obtained by treating surfaces of substrates by using the compound 1-3, the compound 2-1, the compound 3-1, the compound 4-1, the compound 5-1, the compound 6-3, the compound 7-1, and the compound 8-1, respectively. A wet-coating method was used as the surface treatment method.

30 g of silicon oxide was disposed as a vapor-deposition source on a copper hearth in a vacuum vapor-deposition apparatus (“VTR-350M” manufactured by ULVAC KIKKO Inc.). A glass substrate was disposed in the vacuum vapor-deposition apparatus, and the vacuum vapor-deposition apparatus was evacuated of air so that the pressure inside the apparatus became 5×10⁻³ Pa or lower. A substrate including a silicon oxide layer having a thickness of about 20 nm was manufactured by heating the aforementioned hearth to about 2,000° C. and thereby vacuum-depositing silicon oxide on the surface of the substrate.

The substrate including the silicon oxide layer was placed on the sample stage of a spray coater (“API-90RS manufactured by APEIROS) in such a manner that the silicon oxide layer faces upward. Next, 13 g of a heptane solution comprising 0.2 mass % of the compound obtained in each of the above-described examples was charged into the syringe of the spray coater and was sprayed and applied at an atomization pressure of 130 kPa, a distance between the nozzle and the sample surface of 50 mm, and a scanning speed of 300 mm/sec (wet-coating method). After that, the substrate including silicon oxide layer, of which the compound had been applied to the surface, was heat-treated at 140° C. for 30 minutes. As a result, for each of Examples 1 to 8, an evaluation sample (article), in which the substrate, the silicon oxide layer, and the surface layer were laminated in this order, was obtained.

### <Water Repellency>

About 2 μL of distilled water was added dropwise on the surface-treated layer of the article, and the initial water contact angle was measured by using a contact angle measuring apparatus (product name: DM-500 manufactured by Kyowa Interface Science Co., Ltd). An average value of values measured at five points on the surface-treated layer was defined as the water contact angle. The water repellency of the surface-treated layer was evaluated based on criteria shown below. Note that a 20 method was used for the calculation of the water contact angle.

A: The initial water contact angle is 105° or larger.

B: The initial water contact angle is smaller than 105°.

### <Abrasion Resistance (Steel Wool)>

A steel wool Bonstar (#0000) was reciprocated 10,000 times on the surface-treated layer at a pressure of 98.07 kPa and a speed of 320 cm/min according to JIS L0849:2013 (corresponding ISO: 105-X12:2001) by using a reciprocating traverse tester (manufactured by KNT), and then, the water contact angle was measured. The method for measuring the water contact angle after the friction test was the same as that for the initial contact angle in the method for evaluating the water repellency. The smaller the decrease in the water repellency (water contact angle) after the friction is, the smaller the decrease in the performance caused by the friction, and hence the more excellent the abrasion resistance is. The evaluation criteria are as follows.

Decrease in water contact angle=(Initial water contact angle)−(Water contact angle after friction)
A: The decrease in water contact angle after 10,000 reciprocations is 5° or smaller
B: The decrease in water contact angle after 10,000 reciprocations is larger than 5° and is 10° or smaller
C: The decrease in water contact angle after 10,000 reciprocations is larger than 10°

### <Fingerprint Removal Property>

A weight of 1 kg equipped with a red rubber stopper having a diameter of 2 cm, which served as a fingerprint stamp part, was prepared. Next, 70 μL of an artificial fingerprint liquid (manufactured by ISEKYU) was dropped onto a wiping cloth, and the fingerprint stamp was made to adhere to the artificial fingerprint liquid for 1 minute. The fingerprint stamp was made to adhere to a new wiping cloth for 20 seconds in order to remove an excessive artificial fingerprint liquid that had adhered to the fingerprint stamp. After that, an article in which a surface-treated layer was formed was placed on a hot plate of which the temperature was adjusted to 23° C. The fingerprint stamp was stamped on the surface-treated layer. The article to which the artificial fingerprint liquid adhered was placed on a sliding apparatus (product name “HHS-2000” manufactured by Shinto Scientific Co., Ltd.). A planar indenter having an area of 1 cm square was stuck on a wiping cloth (Savina Minimax manufactured by KB Seiren Ltd.) by using a double-sided tape, and the wiping cloth with the indenter stuck thereon was placed on the sliding apparatus. The artificial fingerprint liquid adhered to the surface-treated layer was wiped off by moving the wiping cloth thereon in one direction with a load of 100 g. The haze of the wiped area was measured with a haze meter (product name “NDH7000SP” manufactured by Denshoku Industries Co., Ltd.). The evaluation criteria were as follows.
A: The haze value is lower than 1%
B: The haze value is 1% or higher

### <Light Stability>

The surface-treated layer was irradiated with a light beam (650 W/m², 300 to 700 nm) at a black-panel temperature of 63° C. for 500 hours by using a tabletop xenon arc lamp-type accelerated light-stability testing machine (SUNTEST XLS+: product name, manufactured by Toyo Seiki Kogyo Co. Ltd.), and then the water contact angle of the surface-treated layer was measured by the method described above. The smaller the decrease in the water contact angle after the accelerated light-stability test is, the smaller the decrease in the performance due to the light is, and hence the more excellent the light stability of the surface-treated layer is. The evaluation criteria are as follows.

$$Decrease in water contact angle = ( Initial water contact angle ) - ( Water contact angle after accelerated light - stability test )$$

A: The decrease in water contact angle after the accelerated light-stability test is 3° or smaller
B: The decrease in water contact angle after the accelerated light-stability test is larger than 3° and is 5° or smaller
C: The decrease in water contact angle after the accelerated light-stability test is larger than 5°

TABLE 1

| Example | | 1 | 2 | 3 | 4 | 5 | 6 | 7 | 8 |
|---|---|---|---|---|---|---|---|---|---|
| Compound | | 1-3 | 2-1 | 3-1 | 4-1 | 5-1 | 6-3 | 7-1 | 8-1 |
| Evaluation | Initial water contact | A | A | A | A | A | A | A | A |
| | angle | | | | | | | | |
| | Abrasion resistance | A | A | A | A | A | A | A | A |
| | Fingerprint removal | A | A | A | A | A | A | A | A |
| | property | | | | | | | | |
| | Light stability | A | A | A | A | A | A | C | C |

As shown in Table 1, it has been found that the articles of Examples 1 to 6, in which the compound 1-3, the compound 2-1, the compound 3-1, the compound 4-1, the compound 5-1, and the compound 6-3, which are represented by Formula (1), were respectively used as surface treatment agents are excellent in the light stability compared to the articles of Examples 7 and 8.

A compound according to the present disclosure is useful as a surface treatment agent. Such a surface treatment agent can be used, for example, for substrates in display devices such as touch panel displays, optical elements, semiconductor elements, building materials, automobile components, and nanoimprinting technologies. Further, such a surface treatment agent can be used for bodies, window glasses (front glasses, side glasses, and rear glasses), mirrors, bumpers, and the like in transportation apparatuses such as trains, automobiles, ships, and airplanes. Further, such a surface treatment agent can be used for exterior walls of buildings, tents, photovoltaic modules, sound insulating plates, and outdoor articles such as concrete; and fishing nets, sweep nets, and water tanks. Further, such a surface treatment agent can be used for kitchens, bathrooms, washstands, mirrors, and toiletry components; chandeliers and ceramics such as tiles; and artificial marble and various indoor apparatuses such as air conditioners. Further, such a surface treatment agent can be used for antifouling treatments of jigs, inner walls, pipes, and the like in factories. Further, such a surface treatment agent can be used for goggles, glasses, helmets, pachinko, fibers, umbrellas, play equipment, and soccer balls. Further, such a surface treatment agent can be used as adherents for various packaging materials such as packaging materials for foods, packaging materials for cosmetics, and the interior of pots. Further, such a surface treatment agent can be used for car navigation systems, mobile phones, smart phones, digital cameras, digital video cameras, PDAs, portable audio players, car audios, game apparatuses, lenses for eyeglasses, camera lenses, lens filters, sunglasses, medical apparatuses such as gastroscopes, copy machines, PCs, displays (e.g., liquid crystal displays, organic EL displays, plasma displays, and touch panel displays), touch panels, protective films, and optical components such as antireflection films.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A compound represented by below-shown Formula (1) or Formula (2) 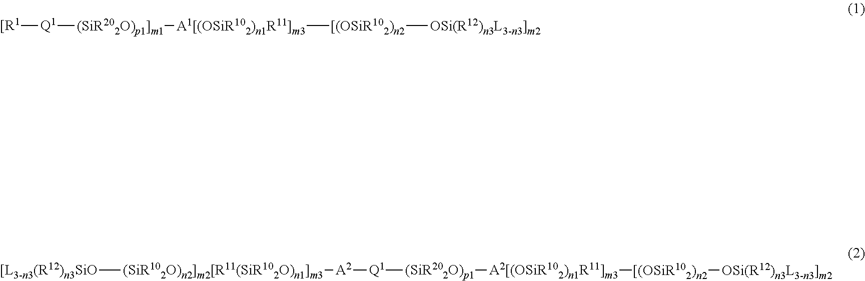
where
R¹ is each independently (R¹¹¹)₃Si—, a monovalent cyclic polysiloxane residue, or a monovalent cage-like polysiloxane residue,
R¹¹¹ is each independently a hydrocarbon group or a trialkylsilyloxy group, and Q¹ is each independently an oxygen atom or an alkylene group,
A¹ is a silicon atom, a cyclic organopolysiloxane residue, or a cage-like organopolysiloxane residue, and is a group having a valence of (m1+m2+m3),
A² is a silicon atom, a cyclic organopolysiloxane residue, or a cage-like organopolysiloxane residue, and is a group having a valence of (1+m2+m3),
R¹⁰ is each independently a hydrocarbon group,
R¹¹ is each independently a hydrocarbon group,
R¹² is each independently a hydrocarbon group,
R²⁰ is each independently a hydrocarbon group,
p1 is a number of 1 to 500,
L is each independently a hydrolyzable group, a group having a hydrolyzable group, or a hydroxyl group,
n1 is each independently an integer of 0 to 6,
n2 is each independently an integer of 0 to 6,
n3 is an integer of 0 to 2,
m1 is an integer of 1 or greater,
m2 is an integer of 2 or greater, and
m3 is an integer of 0 or greater.

2. The compound according to claim 1, wherein Q¹ is an oxygen atom.

3. The compound according to claim 1, wherein m1 is an integer of 1 to 6.

4. The compound according to claim 1, wherein m2 is an integer of 2 to 6.

5. The compound according to claim 1, wherein m3 is 0 or 1.

6. The compound according to claim 1, wherein A¹ is a residue obtained by excluding (m1+m2+m3) pieces of R from below-shown Formula (A1), Formula (A2), or Formula (A3) 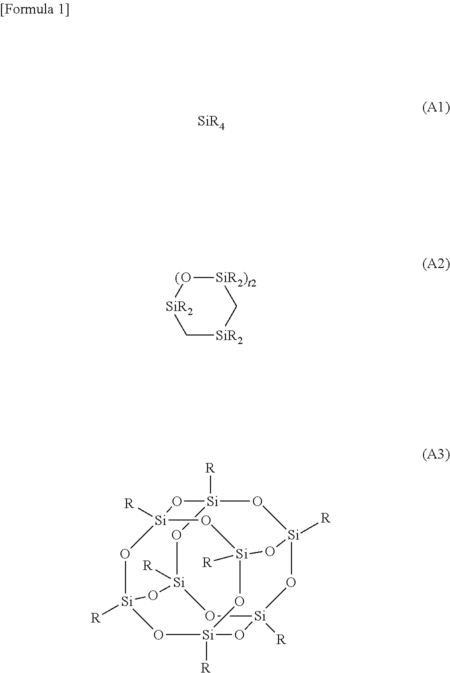
where
R is each independently is a hydrocarbon group, or indicates a position of a bond with [R¹-Q¹-(SiR²⁰₂O)ₚ₁], [(OSiR¹⁰₂)ₙ₁R¹¹], or [(OSiR¹⁰₂)ₙ₂—OSi(R¹²)ₙ₃L₃₋ₙ₃] in Formula (1), and
t2 is an integer of 1 to 4.

7. The compound according to claim 1, wherein A² is each independently a residue obtained by excluding (1+m2+m3) pieces of R from below-shown Formula (A1), Formula (A2), or Formula (A3) 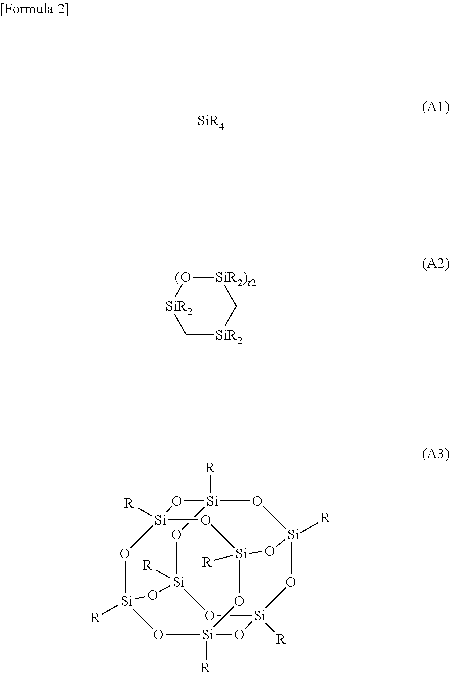
where
R is each independently a hydrocarbon group, or indicates a position of a bond with [(OSiR¹⁰₂)ₙ₁R¹¹] or [(OSiR¹⁰₂)ₙ₂—OSi(R¹²)ₙ₃L₃₋ₙ₃] in Formula (2), and
t2 is an integer of 1 to 4.

8. A composition comprising a compound according to claim 1 and a liquid medium.

9. A surface treatment agent comprising a compound according to claim 1.

10. A surface treatment agent comprising a compound according to claim 1 and a liquid medium.

11. A method for manufacturing an article including a surface-treated layer formed on a substrate by performing a surface treatment on the substrate by using a surface treatment agent comprising a compound according to claim 1.

12. An article comprising a substrate, and a surface-treated layer disposed on the substrate, a surface of the surface-treated layer being treated with a surface treatment agent comprising a compound according to claim 1.

13. The article according to claim 12, wherein the article is an optical member.

14. The article according to claim 12, wherein the article is a display or a touch panel.

15. A method for manufacturing an article including a surface-treated layer formed on a substrate by performing a surface treatment on the substrate by using a surface treatment agent comprising a compound according to claim 1 and a liquid medium.

16. An article comprising a substrate, and a surface-treated layer disposed on the substrate, a surface of the surface-treated layer being treated with a surface treatment agent comprising a compound according to claim 1 and a liquid medium.

17. The article according to claim 16, wherein the article is an optical member.

18. The article according to claim 16, wherein the article is a display or a touch panel.
